(19) United States
(12) Patent Application Publication
(10) Pub. No.: US 2022/0111385 A1
(43) Pub. Date: Apr. 14, 2022

(54) SYSTEM AND METHOD FOR BLOOD SAMPLE COLLECTION AND PROCESSING

(71) Applicant: DH Diagnostics LLC, Brea, CA (US)

(72) Inventors: David L. YANG, Orange, CA (US); Fiona S. ADAIR, Brea, CA (US); Dirk HECKEL, Yorba Linda, CA (US)

(21) Appl. No.: 17/558,846

(22) Filed: Dec. 22, 2021

Related U.S. Application Data
(63) Continuation of application No. PCT/US2020/042505, filed on Jul. 17, 2020.
(60) Provisional application No. 62/876,057, filed on Jul. 19, 2019.
(60) Provisional application No. 62/876,064, filed on Jul. 19, 2019.
(60) Provisional application No. 62/876,074, filed on Jul. 19, 2019.

(51) Int. Cl. B01L 3/00; A61B 5/15; G01N 35/00; B01L 9/06

(52) U.S. Cl. CPC B01L 3/502753; G01N 2035/00811; A61B 5/150343; B01L 3/502761; B01L 3/50273; B01L 3/52; G01N 35/00732; B01L 9/06; B01L 2300/168; B01L 2400/0436; B01L 2300/021; B01L 2300/0809; B01L 2300/0609; B01L 2200/0652; B01L 2200/10; G01N 2035/00831; A61B 5/150755

(57) ABSTRACT

Blood sample collection and processing system (1) using a tube (102) and a holder (104) is described herein. The tube (102) includes a plasma separation system (114) for converting a whole blood sample into a plasma sample without using centrifugation. The plasma separation system (114) may comprise a microfluidic separation system such as a lateral cavity acoustic transducer system (113). The tube (102) is thereafter placed in the holder (104) for analysis regarding the plasma. The results of the analysis may be displayed to the user via a display screen (134) or an indicator of a different kind, to allow the user to determine whether the sample is sufficient with regards to volume, and/or quality for further use. Inasmuch as the analysis system (132) performs a rapid analysis of the plasma sample, the patient is still present and if the sample is insufficient, the healthcare provider can take another whole blood sample, counsel the client regarding the blood sample, or examine the patient further.

### CLAIM OF PRIORITY

This patent application is a continuation of International Patent Application No. PCT/US2020/042505, filed on Jul. 17, 2020, which in turn claims the benefit of priority to U.S. Provisional Application Ser. No. 62/876,057, filed on Jul. 19, 2019; U.S. Provisional Application Ser. No. 62/876,064, filed on Jul. 19, 2019; and U.S. Provisional Application Ser. No. 62/876,074, filed on Jul. 19, 2019. All of the above applications are incorporated by reference herein in their entireties.

### BACKGROUND

Oftentimes it is desirable to perform a full biochemical analysis on a whole blood sample taken from a patient. However, prior to the initiation of the full biochemical analysis, pre-analytical steps are taken to determine sufficiency of the whole blood sample. For example, the plasma of the whole blood sample may contain a large number of interferents rendering the whole blood sample insufficient for full biochemical analysis. The pre-analytical steps, from blood collection, to centrifugation of the sample, to examination is laborious, time-consuming, and error prone. Oftentimes, upon a determination that the whole blood sample is insufficient, the patient has left the healthcare facility and must return to the healthcare facility to provide another whole blood sample.

Further, the current process for pre-analytics of blood testing is manual in nature, which is time-consuming and introduces risk of poor sample quality and handling. A phlebotomist draws blood into a single glass or plastic tube through a rubber topped cap via vacuum or low pressure within the tube. The blood sample is transferred to the lab where a clinician processes the sample through multiple steps before the sample is loaded onto a rack of an analyzer instrument. The clinician needs to centrifuge, de-cap, label, scan, order, and rack the sample. The workflow contains about 10-12 steps. Additionally, each step to the process can vary in length with some steps taking up to 1 hour. These lengthy steps are often cut short due to time constraints which may impact the final results. In the occasion that a patient needs multiple tests, various sample types may need to be prepared by repeating this process; not only is this laborious but it can introduce sample handling errors. If there were a more streamlined approach, clinicians would save a lot of time and results would be more reliable.

Current solutions to the reliability problem are directed to improvements on the tube technology, but no current solutions eliminate the need to manually process the sample. Current solutions only make minor improvements to sample quality. Workflow for labs remain cumbersome, time consuming, and more prone to error than proposed solution.

In some cases, a whole blood sample is provided to a lab where a clinician processes the whole blood sample through multiple steps before the sample or derivatives thereof are loaded onto a rack of an analyzer instrument.

Many diagnostic tests are performed on plasma and/or serum samples rather than a whole blood sample. Plasma and/or serum creation is primarily achieved via centrifugation. For most modest throughput labs, this is done manually, whereas high throughput labs use automation lines containing a centrifugation station. This need to centrifuge is time consuming and usually requires batching. Centrifugation on automation tracks is a driver of reduced reliability of the overall automation system.

Therefore, there is a need in the field for simplified workflow and improved reliability regarding generating plasma and/or serum samples from whole blood. In some instances, allowing for random access of the plasma and/or serum samples while keeping a high throughput is desired. Other needs relate to reducing the amount of time required to achieve a result, reducing the price per test, and increasing the reliability of the plasma and/or serum generation.

### SUMMARY

According to a first aspect, there is provided a collection system. The collection system includes a tube and a holder. The holder is configured to receive the tube therein.

According to a second aspect, there is provided a collection system. The collection system includes a tube having a collection chamber, a plasma separation system, and an analysis pocket. The plasma separation system may be disposed in the collection chamber. The analysis pocket extends from the collection chamber. The analysis pocket is configured to receive therein a unit of plasma provided from the plasma separation system. The collection system further includes a holder configured to draw power from a power supply. The holder includes a display, a tube receptacle, an analysis system, a labeling system, and a transducer. The display is powered by the power supply. The tube receptacle is configured to receive the tube therein. The analysis system is configured to collect a set of data from the unit of plasma disposed in the analysis pocket. The analysis system is configured to actuate the display to display the set of data. The labelling system is configured to label the tube. The transducer is configured to oscillate fluid within a main channel and a lateral cavity of the plasma separation system.

The plasma separation system of the collection system according to the second aspect can convert a whole blood sample into a plasma sample without using centrifugation. The plasma separation system may comprise a microfluidic separation system such as a lateral cavity acoustic transducer system. The tube is thereafter placed in the holder for analysis regarding the plasma. The results of the analysis may be displayed to the user via a display screen or an indicator of a different kind, to allow the user to determine whether the sample is sufficient with regards to volume and/or quality for further use. Inasmuch as the analysis system performs a rapid analysis of the plasma sample, the patient is still present, and if the sample is insufficient, the healthcare provider can take another whole blood sample, counsel the client regarding the blood sample, or examine the patient further.

According to a third aspect, there is provided a method. The method includes placing a blood sample into a tube. The method further includes receiving the tube in a holder. The method further includes oscillating the blood sample in the tube with a transducer. The method further includes, in response to oscillating the blood sample in the tube with the transducer, creating a plasma sample from the blood sample within the tube. The method further includes conducting spectroscopic or imaging analysis on the plasma sample with an analysis system of the holder to derive a sample quality.

According to a fourth aspect, there is provided a device. The device includes a main body, a needle attachment feature, a first chamber, a second chamber, and a separation element. The needle attachment feature is configured to couple a needle element to the main body. The needle attachment feature is configured to transmit a blood sample from the needle element into the main body. The separation element is configured to separate the blood sample into a first portion and a second portion. The separation element is configured to transmit the first portion into the first chamber. The separation element is further configured to transmit the second portion into the second chamber.

The device according to the fourth aspect can streamline a process of collecting a blood sample and separating the blood sample into different portions. In some cases, the separation element of the device can quickly produce plasma without centrifugation.

According to a fifth aspect, there is provided a method. The method includes drawing a blood sample into a device. The method further includes separating the blood sample into a sample of whole blood, a sample of plasma, and a sample of serum. The method further includes transmitting the sample of whole blood into a first chamber connected to the device. The method further includes transmitting the sample of plasma into a second chamber connected to the device. The method further includes transmitting the sample of serum into a third chamber connected to the device.

According to a sixth aspect, there is provided a system. The system includes a device having a main body, a needle attachment feature, and a separation element disposed inside the main body. The needle attachment feature is configured to selectively couple a needle element to the main body. The needle attachment feature is configured to transmit a blood sample from the needle element into the main body. The separation element is configured to receive the blood sample and separate the blood sample into a sample of whole blood, a sample of plasma, and a sample of serum.

The system further includes a first sample tube, a second sample tube and a third sample tube. The first sample tube is removably connected to the device. The first sample tube is configured to receive the sample of whole blood from the device. The second sample tube is removably connected to the device. The second sample tube is configured to receive the sample of plasma from the device. The third sample tube is removably connected to the device. The third sample tube is configured to receive the sample of serum from the device.

According to a seventh aspect, there is provided an analyzer configured to receive a tube containing a blood sample. The analyzer includes a separation system, a transfer element, and a movement system. The transfer element is configured to obtain at least a portion of the blood sample from the tube. The transfer element is configured to deposit the portion of the blood sample into the separation system. The movement system is configured to move the transfer element between the tube and the separation system. The separation system is configured to separate the portion of the blood sample into a separated sample. The separated sample includes one of a plasma sample and a serum sample.

The separation system of the analyzer according to the seventh aspect can create a plasma sample or a serum sample from a whole blood sample without centrifugation. The separation system may comprise a microfluidic separation system such as a Lateral Cavity Acoustic Transducer (LCAT) system or similar. The analyzer including the separation system, the transfer element, and the movement system may at least partially automate a process of analyzing a blood sample in a tube.

According to an eighth aspect, there is provided an analyzer system. The analyzer system includes a cassette and an analyzer. The analyzer is configured to receive a tube containing a blood sample and the cassette. The analyzer includes a separation system, a transfer element, a movement system, and a cassette receptacle. The transfer element is configured to obtain at least portion of the blood sample from the tube. The transfer element is configured to deposit the portion of the blood sample into the separation system. The movement system is configured to move the transfer element between the tube and the separation system. The cassette receptacle is defined by the separation system. The cassette receptacle is configured to removably receive the cassette therein. The separation system is configured to separate the portion of the blood sample into a separated sample via the cassette. The separated sample includes one of a plasma sample and a serum sample.

According to a ninth aspect, there is provided a method of analyzing a blood sample in a tube. The method includes placing the tube into an analyzer. The method further includes transferring at least a portion of the blood sample from the tube into a separation system of the analyzer. The transferring may be performed automatically within the analyzer. The method further includes separating the portion of the blood sample into a separated sample by the separation system. The separated sample is one of a plasma sample and a serum sample. The method further includes transferring the separated sample into an analysis system. The transferring is performed automatically within the analyzer. The method further includes analyzing the separated sample via the analysis system.

According to a tenth aspect, there is provided a cassette configured to be selectively disposed in an analyzer. The cassette includes a main body and at least one separation channel disposed within the main body. Each separation channel is configured to separate at least a portion of a blood sample into a separated sample.

According to an eleventh aspect, there is provided an analyzer system. The analyzer system includes a transducer, a LCAT chip, and an LCAT rack. The LCAT chip is configured to receive a whole blood sample and produce sample in response to simulation by the transducer. The LCAT rack is configured to removably receive the LCAT chip therein. The transducer is configured to simulate the LCAT chip to produce plasma while the LCAT chip is in the LCAT rack.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a diagrammatic view of an exemplary system for blood sample collection and processing, according to the principles of the present disclosure;

FIG. 2 depicts an elevational view of an exemplary smart tube system of the system for blood sample collection and processing of FIG. 1, according to the principles of the present disclosure;

FIG. 3 depicts a diagrammatic view of an exemplary computer architecture for use in the system for blood sample collection and processing of FIG. 1, according to the principles of the present disclosure;

FIG. 4 depicts an elevational view of an exemplary tube of the smart tube system of FIG. 2, according to the principles of the present disclosure;

FIG. 5 depicts an elevational view of an exemplary holder of the smart tube system of FIG. 2, according to the principles of the present disclosure;

FIG. 6A depicts an exemplary lateral cavity acoustic transducer for use in plasma generation, according to the principles of the present disclosure;

FIG. 6B depicts a segment of the lateral cavity acoustic transducer of FIG. 6A, according to the principles of the present disclosure;

FIG. 7A depicts another exemplary lateral cavity acoustic transducer for use in plasma generation, according to the principles of the present disclosure;

FIG. 7B depicts another exemplary lateral cavity acoustic transducer for use in plasma generation, according to the principles of the present disclosure;

FIG. 8 depicts a flowchart of an exemplary method for blood sample collection and processing, according to the principles of the present disclosure;

FIG. 9 depicts a perspective view of an exemplary device for blood collection and separation, according to the principles of the present disclosure;

FIG. 10 depicts a perspective view of the device of FIG. 9 with parts cut away;

FIG. 11 depicts a cross-sectional view of an exemplary rack, according to the principles of the present disclosure;

FIG. 12 depicts a perspective view of the device of FIG. 9 with parts cut away and with an exemplary syringe element incorporated therewith, according to the principles of the present disclosure;

FIG. 13 depicts a side elevational view of another exemplary rack with the device of FIG. 9 disposed therein, according to the principles of the present disclosure;

FIG. 14 depicts a flowchart of an exemplary method for collecting and separating a blood sample, according to the principles of the present disclosure;

FIG. 15 depicts a diagrammatical view of an exemplary analyzer, according to the principles of the present disclosure;

FIG. 16 depicts a diagrammatical view of an exemplary cassette for use in the analyzer of FIG. 15, according to the principles of the present disclosure;

FIGS. 17A-17B depict a perspective view and a top plan view, respectively, of an exemplary integrated handler element for use in plasma generation in the analyzer of FIG. 15, according to the principles of the present disclosure;

FIGS. 18A-18C depict a perspective view, a top plan view, and a side elevational view, respectively, of an exemplary integrated wheel element for use in plasma generation in the analyzer of FIG. 15, according to the principles of the present disclosure;

FIG. 19 depicts a perspective view of an exemplary portion of the analyzer of FIG. 15 along with an exemplary rack with an exemplary sample collection tube disposed therein, according to the principles of the present disclosure;

FIG. 20 depicts a side elevational view of an exemplary sample collection tube with an exemplary LCAT insert disposed therein, according to the principles of the present disclosure;

FIG. 21 depicts a diagrammatical view of a flowchart of an exemplary method of using the analyzer of FIG. 15, according to the principles of the present disclosure;

FIGS. 22A-22D depict various perspective views of exemplary LCAT chips and exemplary LCAT racks for use in an exemplary analyzer system, according to the principles of the present disclosure;

FIGS. 23A-23B depict a perspective view and a side elevation view, respectively, of an exemplary movement system, exemplary loading areas, and exemplary transducer slots of the analyzer system of FIGS. 22A-22D, according to the principles of the present disclosure;

FIG. 24 depicts a perspective view of the movement system, loading areas, and transducer slots of FIGS. 23A-23B;

FIGS. 25A-25B depict top plan views of the movement system, loading areas, and transducer slots of FIGS. 23A-23B;

FIGS. 26A-26B depict a side profile view and a perspective view, respectively, of an exemplary LCAT chip and LCAT rack for use in an exemplary analyzer system, according to the principles of the present disclosure;

FIG. 27A depicts a perspective view of a top of the LCAT chip of FIGS. 26A-26B;

FIG. 27B depicts an elevational view of the LCAT chip of FIGS. 26A-26B;

FIGS. 27C-27D depict perspective views of two exemplary transducer cartridges of the analyzer system of FIGS. 26A-26B, according to the principles of the present disclosure;

FIG. 28A depicts a perspective view of the LCAT rack of FIGS. 26A-26B;

FIG. 28B depicts a perspective view of an exemplary plasma separation rack, according to the principles of the present disclosure;

FIG. 28C depicts an exemplary sample cuvette of the analyzer system of FIGS. 26A-26B, according to the principles of the present disclosure; and

FIGS. 29A-29C depict a top plan view, a perspective view, and an elevational view, respectively, of an exemplary movement system, exemplary loading areas, and an exemplary transducing module of the analyzer system of FIGS. 26A-26B.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Exemplary Blood Sample Collection and Processing System

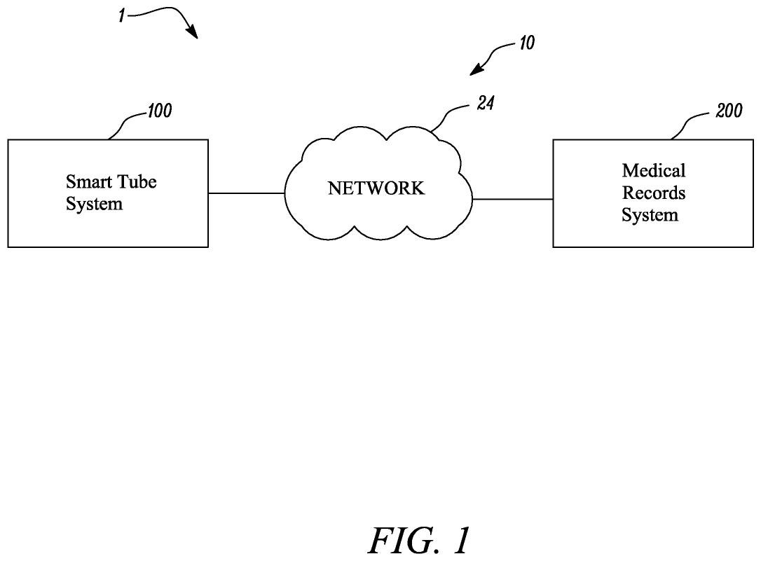

Referring now to FIG. 1, an exemplary blood sample collection and processing system (1) of the present disclosure may include an operating environment (10). In general, blood sample collection and processing system (1) operates to pre-analyze a whole blood sample for sufficiency prior to full biochemical analysis. The pre-analysis is conducted in a rapid manner to provide a sufficiency determination to the healthcare professional regarding the blood sample. For example, a determination may be made while the patient is in the examination room. If the sample is determined to be insufficient, the healthcare professional can counsel the patient regarding the proper fasting or other preparation techniques, redraw another whole blood sample, or examine the patient further.

Blood sample collection and processing system (1) may be applied for testing in locations such as triaging at patient home via emergency medical technicians, to decide if an ambulance and/or hospitalization is required. In ambulance testing may be performed by generating plasma for immediate testing in the ambulance. Communication systems and/or information technology systems may be incorporated with blood sample collection and processing system (1) to allow information and test results to be communicated to the emergency room, hospital, or care provider prior to the arrival of the patient. A virtual doctor could also be used in conjunction with blood sample collection and processing system (1).

In other environments and applications, blood sample collection and processing system (1) may be used in self-testing to prevent impaired operation of machinery such as cars, aircraft, or manufacturing. Other applications may relate to bar testing to prevent liability to the bar; athlete testing to identify doping; rehabilitation centers to monitor patients; prison and probation situations to monitor inmates; schools to monitor teachers and students; drug testing for employment and compliance; drug interactions with drugs of abuse (“DOA”) as current DOA testing is done based on urine, breath, saliva, and/or hair analysis; horse, dog, and/or camel racing and other doping opportunities; home testing for teens or other high risk groups; welfare recipients; jury selection; automatic safety system for vehicle lockout; and/or truck driver monitoring.

In addition to human blood testing, there is a need to test animal blood in a variety of settings. For example, there is a need to test animal blood in veterinarian offices; in large animal clinics; in small animal clinics; in animal production facilities such as chicken farms, in food production, in meat/food quality testing; in zoos; in nature preserves for wild animals; for tagged animals; in U.S.D.A. facilities and/or government animal regulatory facilities; in ports of entry for disease testing; in quarantine; in animal health monitoring for antibody production; in animal health monitoring for diagnostic controls; in research, university, and/or industry space; in vaccine testing; in cosmetic testing; in drug and/or pharmaceutical testing; in testing for rabies; in animal shelters; in pet stores; in grooming pet spas such as for a quick pet check during a grooming session. Providing a quick way to initiate a biochemical analysis such as through blood sample collection and processing system (1) may provide remote access to testing at farms, wildlife refuges, zoos, or other environments. A simple method for biochemical testing such as via blood sample collection and processing system (1) allows lower skilled personnel to conduct the testing. Current testing for animals is done by sending samples out to large reference labs or large veterinarian clinics. A faster turnaround time such as the turnaround time provided by blood sample collection and processing system (1) may increase test usage and provide faster results for emotional pet owners. Blood sample collection and processing system (1) may aid in standardizing hematocrit in animals. Animals with very little blood such as avian animals may be processed via blood sample collection and processing system (1) due to the need for less volume of whole blood.

With respect to field testing for health monitoring, fatigue testing, and dosing, blood sample collection and processing system (1) may provide accurate measurements quickly for both monitoring and trauma, such as for soldiers; sailors; endurance athletes; astronauts; truck drivers; machine operators; pilots; and diabetic, thyroid, and/or allergy patients. Areas of low resources such as remote areas or in disaster response environments may benefit from in-field testing using blood sample collection and processing system (1).

The ability to quickly generate plasma without large, expensive, and/or complicated instrumentation such as centrifugation may be addressed by the present disclosure. For certain tests, plasma testing is often likely to give a more accurate and more sensitive result than whole blood testing. The blood sample collection and processing system (1) may also enable more sensitive and/or specific solution-based testing when compared to current practice.

The amount of plasma needed for a given test based on test orders may be calculated and incorporated into the whole blood draw requirements. A phlebotomist may be guided to the right type, number, and tube size for the sample draws. The sample stability (aging) and sample quality may be monitored by blood sample collection and processing system (1). The sample quality may also be provided to a central analysis platform for tracking, tracing, and auditing the plasma generation system or any other components of blood sample collection and processing system (1).

The blood collection and processing may be provided through a smart tube system (100) and a medical records system (200). In some versions of operating environment (10), smart tube system (100) and medical records system (200) may send and receive communications data between one another directly. Alternatively, in other versions of operating environment (10), smart tube system (100) and medical records system (200) may communicate with each other through a network (24). Network (24) may include one or more private or public networks (e.g., the Internet) that enable the exchange of data. In yet other versions of operating environment (10), medical records system (200) is omitted.

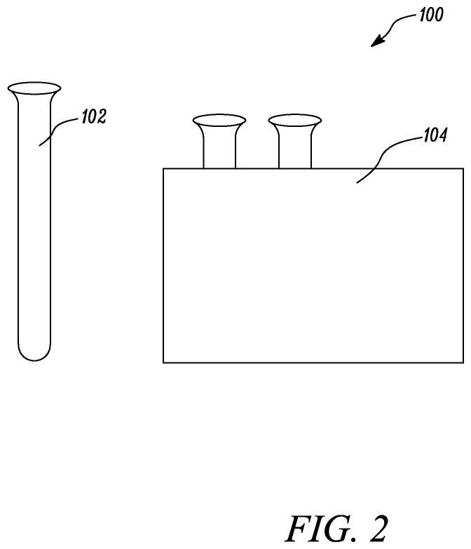

As shown in FIG. 2, smart tube system (100) includes a tube (102) and a holder (104). In general, tube (102) is configured to receive a whole blood sample therein and generate a plasma sample from the blood sample within tube (102). The plasma sample is generated in a rapid manner without using centrifugation and may be accomplished with a small whole blood sample. For example, in some versions of smart tube system (100), a plasma sample may be generated from less than 300 microliters (μL) of whole blood.

Once tube (102) contains a whole blood sample, tube (102) is thereafter placed in holder (104) for analysis of the blood sample and/or plasma sample. The medical records of the patient associated with the blood sample may be accessed through medical records system (200) by holder (104) to aid in analyzing the plasma sample or for use in labeling tube (102). Conversely, holder (104) may provide information regarding the patient's plasma sample to medical records system (200) for storage therein.

A. Exemplary Computer System

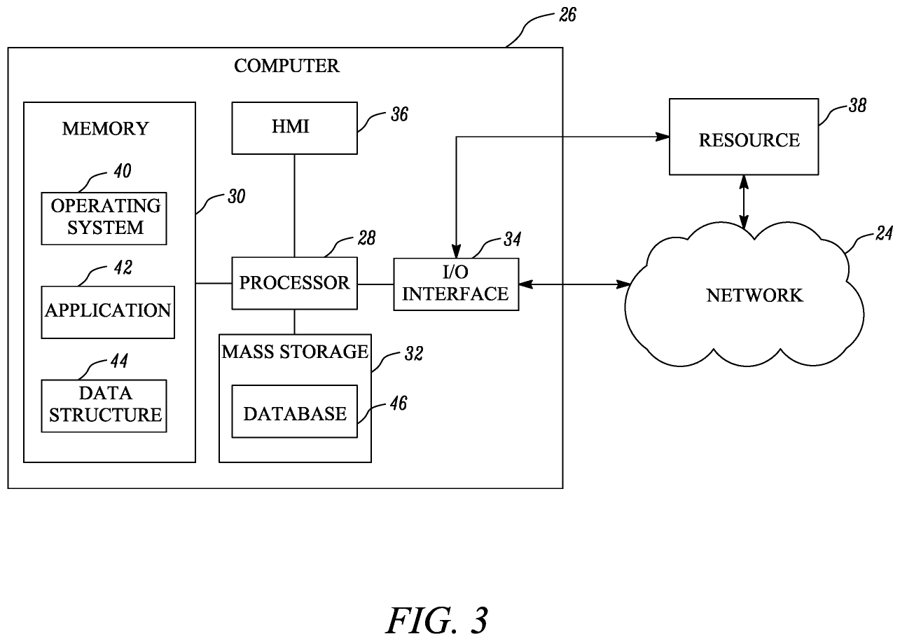

Referring now to FIG. 3, all or portions of smart tube system (100), medical records system (200), and network (24) of operating environment (10) may be implemented on one or more computing devices or systems, such as an exemplary computer system (26). Computer system (26) may include a processor (28), a memory (30), a mass storage memory device (32), an input/output (I/O) interface (34), and a Human Machine Interface (HMI) (36). Computer system (26) may also be operatively coupled to one or more external resources (38) via network (24) or I/O interface (34). External resources may include, but are not limited to, servers, databases, mass storage devices, peripheral devices, cloud-based network services, or any other suitable computer resource that may be used by computer system (26).

Processor (28) may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in memory (30). Memory (30) may include a single memory device or a plurality of memory devices including, but not limited, to read-only memory (ROM), random access memory (RAM), volatile memory, non-volatile memory, static random-access memory (SRAM), dynamic random access memory (DRAM), flash memory, cache memory, or any other device capable of storing information. Mass storage memory device (32) may include data storage devices such as a hard drive, optical drive, tape drive, non-volatile solid-state device, or any other device capable of storing information.

Processor (28) may operate under the control of an operating system (40) that resides in memory (30). Operating system (40) may manage computer resources so that computer program code embodied as one or more computer software applications, such as an application (42) residing in memory (30), may have instructions executed by processor (28). In an alternative embodiment, processor (28) may execute the application (42) directly, in which case operating system (40) may be omitted. One or more data structures (44) may also reside in memory (30), and may be used by processor (28), operating system (40), or application (42) to store or manipulate data.

I/O interface (34) may provide a machine interface that operatively couples processor (28) to other devices and systems, such as network (24) or external resource (38). Application (42) may thereby work cooperatively with network (24) or external resource (38) by communicating via I/O interface (34) to provide the various features, functions, applications, processes, or modules comprising embodiments of the disclosure. Application (42) may also have program code that is executed by one or more external resources (38), or otherwise rely on functions or signals provided by other system or network components external to computer system (26). Indeed, given the nearly endless hardware and software configurations possible, persons having ordinary skill in the art will understand that embodiments of the disclosure may include applications that are located externally to computer system (26), distributed among multiple computers or other external resources (38), or provided by computing resources (hardware and software) that are provided as a service over network (24), such as a cloud computing service.

HMI (36) may be operatively coupled to processor (28) of computer system (26) in a known manner to allow a user to interact directly with computer system (26). HMI (36) may include video or alphanumeric displays, a touch screen, a speaker, and any other suitable audio and visual indicators capable of providing data to the user. HMI (36) may also include input devices and controls such as an alphanumeric keyboard, a pointing device, keypads, pushbuttons, control knobs, microphones, etc., capable of accepting commands or input from the user and transmitting the entered input to processor (28).

A database (46) may reside on mass storage memory device (32), and may be used to collect and organize data used by the various systems and modules described herein. Database (46) may include data and supporting data structures that store and organize the data. In particular, database (46) may be arranged with any database organization or structure including, but not limited to, a relational database, a hierarchical database, a network database, or combinations thereof. A database management system in the form of a computer software application executing as instructions on processor (28) may be used to access the information or data stored in records of database (46) in response to a query, where a query may be dynamically determined and executed by operating system (40), other applications (42), or one or more modules.

B. Exemplary Tube

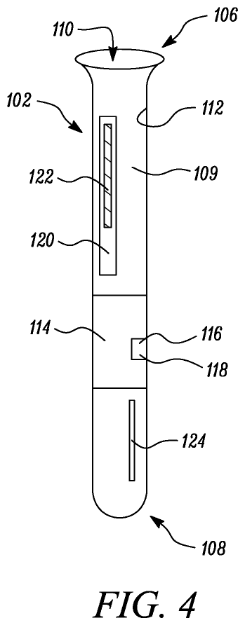

As shown in FIG. 4, tube (102) extends from a top end (106) to a bottom end (108) and includes an exterior surface (109). A top opening (110) is defined at top end (106). Top opening (110) opens to a collection chamber (112) defined within tube (102) to allow for the blood sample to be placed inside tube (102).

A plasma separation system (114) is disposed within collection chamber (112). Plasma separation system (114) is configured to filter, convert, or otherwise extract a portion of the blood sample disposed in collection chamber (112) into a plasma sample without using centrifugation. In some versions of tube (102), plasma separation system (114) may comprise a microfluidic separation system such as a lateral cavity acoustic transducer system. Other versions of tube (102) may incorporate various features from a lateral cavity acoustic transducer system into plasma separation system (114).

Plasma separation system (114) acts to filter out or otherwise extract plasma from the whole blood placed into collection chamber (112). In some versions of plasma separation system (114), the non-plasma elements of the filtered whole blood sample travels through plasma separation system (114) and is deposited proximate bottom end (108) of tube (102), while the plasma sample is maintained within plasma separation system (114). Plasma separation system (114) may be configured to separate the plasma sample from the whole blood sample in a rapid manner. In some versions of plasma separation system (114), the plasma sample may be generated in under ten minutes.

Collection chamber (112) may define an analysis pocket (116). Analysis pocket (116) operates cooperatively with plasma separation system (114) to receive a unit of the plasma sample therein. Tube (102) includes a portion (118) adjacent to analysis pocket (116). In some versions of tube (102), portion (118) is transparent to allow for sufficient optical imaging of the unit of the plasma sample therein.

On exterior surface (109) of tube (102), a label portion (120) may be defined and configured to receive a label (122) thereon. Exterior surface (109) of label portion (120) may include a different physical property from the rest of exterior surface (109). For example, exterior surface (109) of label portion (120) may include a textured surface to aid in receiving and adhering label (122) thereon. Exterior surface (109) of label portion (120) may be a flattened or planer area, rather than the typical angular or rounded surface on other portions of tube (102) to aid in receiving label (122). Label (122) may alternatively comprise an electronic label such as an RFID chip programmable to include patient information or other relevant information therein.

Alternatively, labeling may be applied directly to the outer surface of tube (102) by etching or otherwise imprinting onto tube (102). In another example, a pre-applied substrate already on tube (102) may be imprinted upon.

In some versions of tube (102), an alignment element (124) may extend from exterior surface (109). Alignment element (124) may be a fin, protuberance, flange or an otherwise “male” style element for aligning tube (102) in a particular orientation within a corresponding “female” style element of holder (104). Conversely, alignment element (124) may be a female style element, while holder (104) may provide the corresponding male element. In other versions of tube (102), alignment element may be another style of mechanism for positioning tube (102) in a desired orientation within holder (104). For example, a magnetic connection may be provided to align tube (102) or any other commonly used features for positioning tube (102) in a particular orientation with respect to holder (104).

C. Exemplary Holder

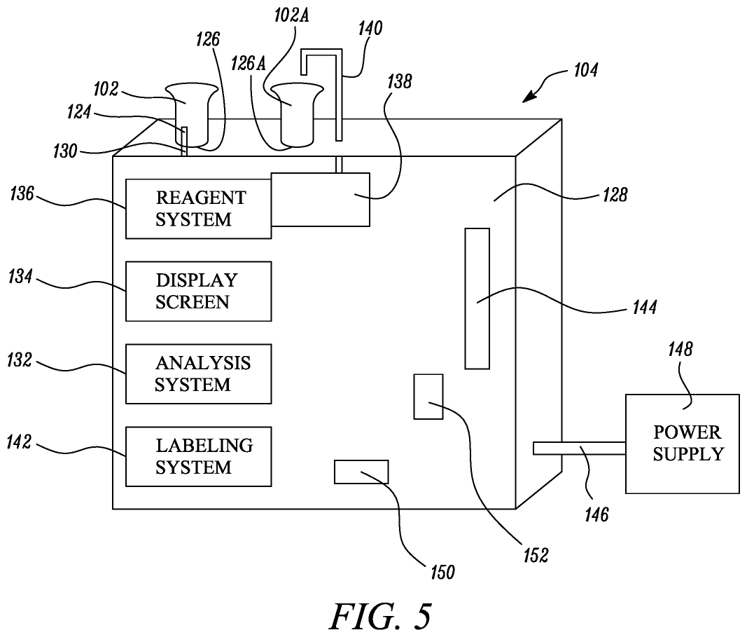

As shown in FIG. 5, holder (104) is configured to receive tube (102) in a tube receptacle (126) defined by a body (128) of holder (104). An alignment element (130) is associated with tube receptacle (126) and configured to cooperate with alignment element (124) of tube (102) to position tube (102) in a particular orientation within holder (104). In some versions of blood sample collection and processing system (1), alignment element (124) of tube (102) is one of a male or female element and alignment element (130) of holder (104) is the other one of the male or female element. For example, alignment element (124) may be a fin extending from exterior surface (109) of tube (102), while alignment element (130) may be a recess or notch associated with tube receptacle (126) and sized to receive the fin of alignment element (124) therein.

Holder (104) may be configured to receive multiple tube elements therein. For example, tube (102) and tube (102A) may both be disposed within a corresponding tube receptacle (126) and tube receptacle (126A), respectively. Different versions of holder (104) may be configured to receive different numbers of tubes (102).

Some versions of holder (104) include an analysis system (132) configured to collect a set of data from the unit of plasma disposed in analysis pocket (116) once tube (102) is disposed in holder (104). Analysis system (132) may include various optical features such as a spectroscopic element for conducting an optical analysis of the unit of plasma. Analysis system (132) is configured to rapidly analyze the whole blood and/or unit of plasma. The rapid analysis takes place while the patient is still in the examination room to allow immediate action if sample quality is poor. With the patient still in the examination room, the healthcare professional can either redraw a sample of blood from the patient, counsel the patient regarding the issues with the blood sample, or examine the patient further.

For example, analysis system (132) may determine the quality of plasma to ensure acceptable levels of interferents such as hemoglobin, lipids, or bilirubin. If unacceptable levels of these interferents are present, the healthcare professional may counsel the patient, who is still in the examination room, regarding the necessary steps to take to ensure the next sample is acceptable (e.g., fast longer, refrain from eating fatty foods prior to the blood draw, etc.), redraw the sample of blood, or examine the patient further. Analysis system (132) may generate a capillary serum index for use in determining whether the whole blood sample is sufficient. Methods for generating a capillary serum index are disclosed in U.S. Pat. Nos. 9,007,574 and 9,588,038, which are incorporated herein by reference.

Some versions of holder (104) include a display screen (134). Display screen (134) may provide a graphical user interface similar to I/O interface (34) as described above. In some versions of holder (104), display screen (134) is actuated by analysis system (132) to display information related to the set of data collected from the unit of plasma disposed in analysis pocket (116) and analyzed by analysis system (132). This information may include hemoglobin, lipid, or bilirubin levels.

Inasmuch as analysis system (132) is configured to provide a rapid analysis of the unit of plasma, in some scenarios, the patient is still in the examination room when the analysis is completed. In response to reviewing the information, the healthcare professional may determine the sample of whole blood is insufficient for a particular purpose and counsel or advise the patient accordingly. For example, analysis of the unit of plasma via analysis system (132) may indicate poor quality plasma, with unacceptable levels of interferents such as hemoglobin, lipids, or bilirubin.

In some versions, holder (104) includes a reagent system (136). Reagent system (136) includes a reservoir (138) filled with one or more reagents for use in mixing with the whole blood sample or the plasma sample to isolate or indicate the presence or absence of a particular element. Reagent system (136) includes corresponding deposit element (140) comprised of tubing, channels, and/or a nozzle extending from body (128) of holder (104) for use in delivering the selected reagent into collection chamber (112) of tube (102) once tube (102) is disposed in holder (104).

For example, the healthcare professional may desire to determine an amount of a particular protein in a whole blood sample. After placing tube (102) into holder (104), the healthcare professional actuates an interface on display screen (134) to actuate reagent system (136) to transfer a deposit of “Reagent A” from reservoir (138), through deposit element (140) and into collection chamber (112) of tube (102). Once Reagent A is mixed with the whole blood sample, analysis system (132) determines the amount of a particular protein in the whole blood sample and provides this information on display screen (134) for review by the healthcare professional.

In some versions, holder (104) includes a labeling system (142). Labeling system (142) is configured to apply label (122) to label area (120) of tube (102). In some versions of blood sample collection and processing system (1), smart tube system (100) receives information regarding the patient from medical records system (200) and applies this information to label (122). For example, identification information for the patient may be received from medical records system (200), formatted, and applied to label (122) by labeling system (142). Thereafter, tube (102) includes the patient identification system. In some versions of labeling system (142), information may be converted into a machine-readable format such as a bar code or QR code for application onto label (122).

Labeling system (142) may incorporate data supplied by the healthcare professional through display screen (134) into the information printed on label (122). The patient's contact information, date of examination, the healthcare professional's initials, or any other information may be entered by the healthcare professional into display screen (134) and transferred onto label (122). Labeling system (142) may include a laser or inkjet style printing element for transferring information onto label (122). In other versions of labeling system (142), the information is printed or transferred directly onto tube (102) forgoing label (122). This may be achieved by etching or otherwise imprinting the information onto label area (120) directly. In other versions of labeling system (142), label (122) incorporates a radio-frequency identification tag technology into the physical body of label (122) to allow for location tracking and passive information transmissions from label (122) regarding tube (102).

In those versions of label (122) embodied in an electronic label such as an RFID chip, labeling system (142) is configured to program label (122) with patient data or other relevant information.

Current labeling solutions rely on a healthcare professional manually transferring a handwritten or computer-generated label from onto a tube. Mislabeled samples lead to incorrect testing, incorrect diagnosis, and incorrect treatment. Studies indicate the financial effect of a mislabeled sample is hundreds of dollars and the emotional impact is incalculable. Further, correct placement of a label onto a tube is important for sample collection. A wrinkled, twisted, non-horizontal, incorrectly oriented label, or a label which prevents observation of the sample can cause delays in processing the sample or may even require the patient to provide another whole blood sample. Labeling system (142) reduces sample mislabeling by simplifying the workflow for the healthcare professional through automatic application of a label to the corresponding tube (102) in a correct and repeatable placement and orientation.

Power may be provided to holder (104) through a battery (144) or via a power cord (146) connected to a power supply (148). In some versions of holder (104), power supply (148) is used for both charging battery (144) as well as supplying holder (104) with power. In other versions, battery (144) is charged directly through power supply (148) and holder (104) runs off battery (144). Battery (144) may be swappable with another battery (144).

As shown in FIG. 5, communication between holder (104) and medical records system (200) may be facilitated through a wired network connection formed through network jack (150). Network jack (150) is configured to receive one end of a communication cable and may be an “ethernet” style or any other style jack for facilitating wired communications with network (24). Communication between holder (104) and medical records system (200) may be facilitated through a wireless network connection formed through wireless module (152). Wireless module (152) is configured to wirelessly communicate with a corresponding wireless module such as those found in wireless modems for facilitating wireless communication with network (24).

D. Exemplary Lateral Cavity Acoustic Transducer

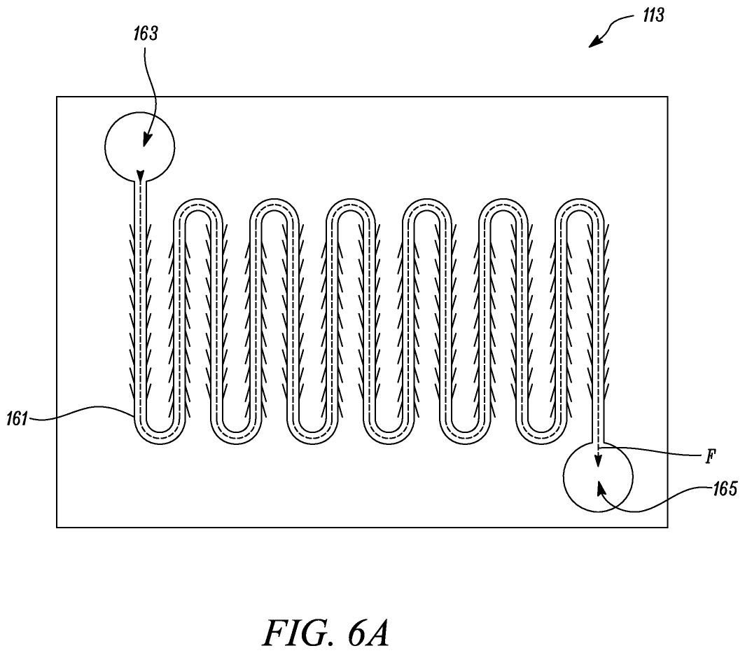

As shown in FIG. 6A, plasma separation system (114) may comprise a microfluidic separation system such as a lateral cavity acoustic transducer system (113) having a microfluidic channel with a plurality of segments, with one segment (115) of lateral cavity acoustic transducer system (113) depicted in FIG. 6B. In general, lateral cavity acoustic transducer system (113) receives as input whole blood and separates the whole blood into plasma and the remainder as the blood moves through the various segments. The relative movement of blood cells and other non-plasma elements is retarded or slowed to allow the plasma of the whole blood to be expelled from lateral cavity acoustic transducer system (113) first, thus separating the plasma from the whole blood for use as needed by smart tube system (100).

In general, lateral cavity acoustic transducer system (113) comprises two parts: a microfluidic channel that contains the blood and plasma; and an acoustic transducer that provides the energy for the oscillations of the blood and plasma within the microfluidic channel. These two parts may be in/on tube (102) or in/on holder (104) in any different configuration. For example, in some versions of lateral cavity acoustic transducer system (113), the microfluidic channel is disposed in tube (102) and the acoustic transducer is disposed in holder (104). In other versions of lateral cavity acoustic transducer system (113), the microfluidic channel is disposed in tube (102) and the acoustic transducer is also disposed in tube (102).

As shown in FIG. 6A, a channel (161) of lateral cavity acoustic transducer system (113) receives a fluid at an inlet (163). The fluid flows through channel (161) along a flow path F and exits channel (161) at an outlet (165). Channel (161) is a microfluidic channel and includes plurality of segments, such as segment (115) depicted in FIG. 6B.

Segment (115) generally extends from a first end (117) to a second end (119), whereby fluid such as whole blood moves in the direction of Arrow A within a main channel (127) from first end (117) to second end (119). A plurality of lateral cavities (121) are filled with trapped air and disposed at various distances along the length of segment (115). Lateral cavities (121) generally extend in a non-orthogonal manner away from main channel (127), as shown in FIG. 6B. Lateral cavity acoustic transducer system (113) further includes a transducer (123) configured to apply an external source of acoustic energy to segment (115) and oscillate air within the lateral cavities (121) and fluid in the main channel (127). Transducer (123) may be a piezo-electric transducer or any other style of transducer or mechanism for oscillation.

Actuation of transducer (123) effectuates oscillation of segment (115) and more specifically, the trapped air within lateral cavities (121). The oscillation of the trapped air causes net flow through segment (115), though as vortices (eddies, whirlpools, etc.) develop within the whole blood, cell flow is retarded. A pair of vortices (125) are depicted in FIG. 6B proximate two lateral cavities (121), which retards the cells within the whole blood moving through segment (115). These vortices (125) retard the flow of the cells and allows the plasma to move ahead of the cell front. The plasma is thereafter expelled from lateral cavity acoustic transducer system (113) before the cells are expelled, effectively separating the plasma from the rest of the whole blood. This allows smart tube system (100) to quickly produce plasma for further use therein without centrifugation.

Transducer (123) may be located within tube (102), within holder (104), or within any other portion or element of smart tube system (100) or blood sample collection and processing system (1).

Main channel (127) of segment (115) has a thickness (171). Thickness (171) may be uniform or may vary along a length of main channel (127). In an example, thickness (171) may be about 500 microns (μm). Main channel (127) may also define a line or plane of symmetry (173). Vortices (125) may be formed on respective sides of line or plane of symmetry (173). Lateral cavities (121) are inclined to main channel (127) by an angle (175). Angle (175) may be uniform or vary across lateral cavities (121). In an example, angle (175) may be about 15 degrees. Angle (175) can also be defined between each lateral cavity (121) and line or plane of symmetry (173).

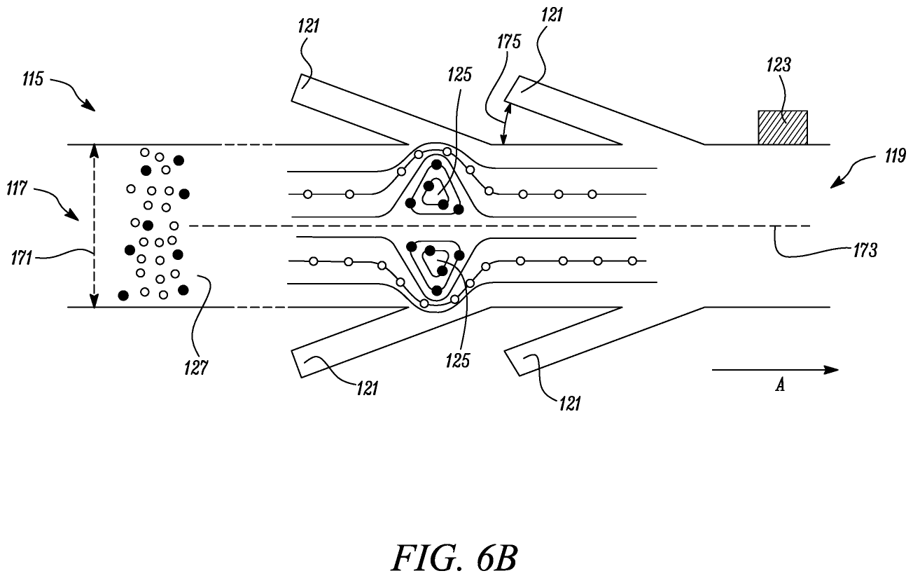

As shown in FIG. 7A, plasma separation system (114) may comprise a lateral cavity acoustic transducer system (213A) similar to lateral cavity acoustic transducer system (113) depicted in FIG. 6A. Lateral cavity acoustic transducer system (213A) generally extends from an inlet (210A) to a measurement cavity (220A). Lateral cavity acoustic transducer system (213A) has a microfluidic channel (214A) with a plurality of segments, similar to segment (115) depicted in FIG. 6B. Lateral cavity acoustic transducer system (213A) further comprises a mixing cavity (215A). Mixing cavity (215A) may be disposed between two segments (115) of lateral cavity acoustic transducer system (213A). Mixing cavity (215A) comprises reagents and mixes separated plasma with the reagents in mixing cavity (215A). The mixed plasma is then transported to measurement cavity (220A). Lateral cavity acoustic transducer system (213A) can therefore generate plasma without centrifugation. Measurement cavity (220A) may enable measurements that need to be performed on plasma instead of whole blood. Such measurements may include glucose measurements and may eliminate the need of saponins for glucose measurements. Methods and reagents for such glucose measurements are disclosed in U.S. Pat. No. 5,278,047, which is incorporated herein by reference.

Fluid generally flows in a direction B inside lateral cavity acoustic transducer system (213A) from inlet (210A) to measurement cavity (220A). Lateral cavity acoustic transducer system (213A) may therefore comprise two segments (115) fluidically connected in a series configuration.

Lateral cavity acoustic transducer system (213A) shown in FIG. 7A is exemplary in nature. In general, lateral cavity acoustic transducer system (213A) may include a plurality of segments (115) fluidically connected in a series configuration and disposed between inlet (210A) and measurement cavity (220A). Adjacent segments (115) may be separated by respective mixing cavity (215A).

As shown in FIG. 7B, plasma separation system (114) may comprise a lateral cavity acoustic transducer system (213B) equivalent to lateral cavity acoustic transducer system (113) depicted in FIG. 6A. Lateral cavity acoustic transducer system (213B) generally extends from an inlet (210B) and splits into three measurement cavities (220B). Lateral cavity acoustic transducer system (213B) has three microfluidic channels (214B) with a plurality of segments, each segment similar to segment (115) depicted in FIG. 6B. Lateral cavity acoustic transducer system (213B) further comprises three mixing cavities (215B). Mixing cavities (215B) may be disposed between two segments (115) of each microfluidic channel of lateral cavity acoustic transducer system (213B). Mixing cavities (215B) comprise respective reagents and mix separated plasma with the respective reagents in mixing cavities (215B). For example, one of mixing cavity (215B) may comprise reagents to provide HDL measurement, one of mixing cavity (215B) may comprise reagents to provide triglycerides measurement, and one of mixing cavity (215B) may comprise reagents to provide total cholesterol measurement. The mixed plasma is then transported to respective measurement cavities (220B). Measurement cavities (220B) may enable measurements that need to be performed on plasma instead of whole blood. Such measurements may include lipid measurements. Devices and methods for such lipid measurements are disclosed in U.S. Pat. No. 9,347,958, which is incorporated herein by reference.

Fluid generally flows in a direction C inside individual microfluidic channels (214B) of lateral cavity acoustic transducer system (213B) from inlet (210B) to respective measurement cavities (220B). Each microfluidic channel (214B) of lateral cavity acoustic transducer system (213B) may therefore comprise two segments (115) fluidically connected in a series configuration. Further, microfluidic channels (214B) are disposed in a parallel configuration with respect to each other.

Lateral cavity acoustic transducer system (213B) shown in FIG. 7B is exemplary in nature. In general, lateral cavity acoustic transducer system (213B) may include a plurality of microfluidic channels (214B) disposed in a parallel configuration with respect to each other. Each microfluidic channel (214B) extends from inlet (210B) to respective measurement cavities (220B). Each microfluidic channel (214B) includes a plurality of segments (115) fluidically connected in a series configuration. Adjacent segments (115) may be separated by respective mixing cavity (215B).

Lateral cavity acoustic transducer system (213B) can generate plasma without centrifugation. In an example, lateral cavity acoustic transducer system (213B) may be incorporated in a cuvette for performing measurements. The cuvette may be inserted in an analyzer. The analyzer may include various components for performing measurements, such as an array of multi-wavelength Light Emitting Diodes (LED) photodiodes and an array of photodiodes corresponding to respective measurement cavities (220B). The photodiodes may be used to measure a particular range of wavelengths received from respective measurement cavities (220B). For example, a blue color may be measured by absorbance measurement at 660 nanometers (nm) and against a reference wavelength at 880 nm.

Lateral cavity acoustic transducers, described above with reference to FIGS. 6A, 6B, 7A, and 7B, are exemplary in nature. Details of certain lateral cavity acoustic transducers are disclosed in U.S. Pat. No. 9,517,465, which is incorporated herein by reference in its entirety.

II. Exemplary Blood Sample Collection and Processing Method

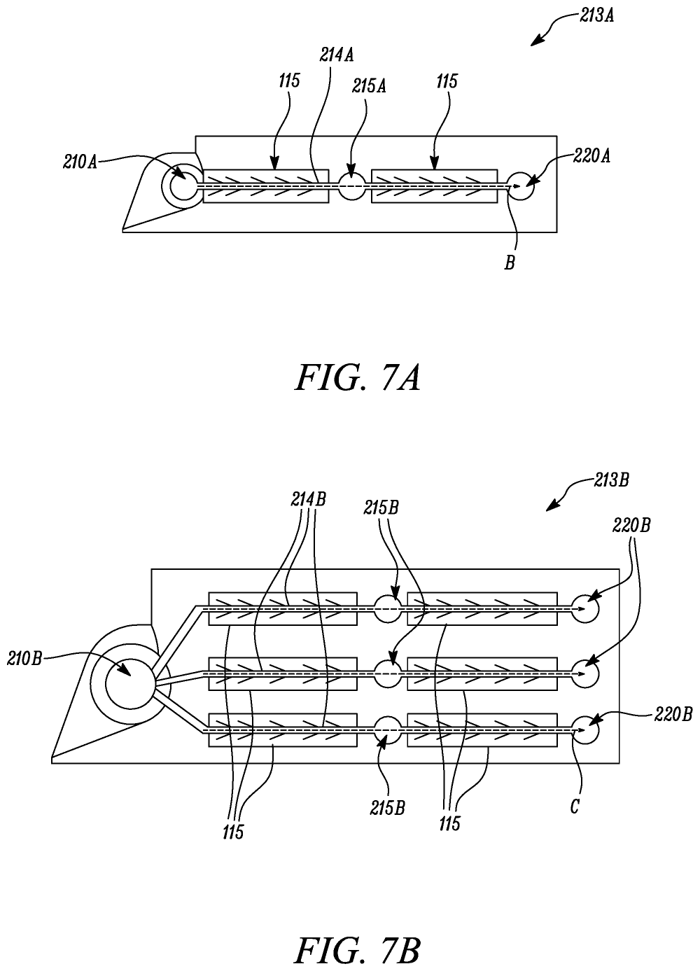

As shown in FIG. 8, a method (300) may be used to collect and process a blood sample. Method (300) begins with a step (302), whereby a blood sample is collected from a patient and placed in a tube. The tube of method (300) may be similar to tube (102) as described above. The blood sample may be withdrawn from the patient directly into the tube, or indirectly withdrawn into an intermediate container and thereafter disposed into the tube. Thereafter, step (302) proceeds to a step (304).

In step (304), a plasma sample is created in the tube from the blood sample disposed in the tube. The mechanism for creating the plasma sample may be in the form of a microfluidic separation system such as a lateral cavity acoustic transducer or a similar system for creating a plasma sample from the blood sample within the tube. Lateral cavity acoustic transducer may apply an acoustic energy to a main channel and associated lateral cavities to retard the movement of cells within the whole blood, leaving the plasma to exit the lateral cavity acoustic transducer system first, thus producing plasma from whole blood without centrifugation. Thereafter, step (304) proceeds to a step (306). In step (306), the user disposes the tube in a holder. The holder of method (300) may be similar to holder (104) as described above. While method (300) is shown with step (304) proceeding step (306), in other versions of method (300), step (306) may proceed step (304), with the user placing the tube into the holder and thereafter the plasma sample created from the blood sample. After the tube is disposed in the holder, step (306) moves to a step (308).

In step (308), the plasma sample in the tube is analyzed by the holder. In some versions of the holder, an analysis system of the holder may be configured to analyze the plasma sample in the tube. The analysis system of method (300) may be similar to analysis system (132) as described above. The analysis system may optically analyze the plasma in the tube to conduct the analysis and to determine whether the plasma sample and/or the blood sample is sufficient or whether an action such as drawing another blood sample is necessary. Inasmuch as the analysis is conducted rapidly, the patient is still in the examination room and readily available for another blood draw or in-person counseling by the healthcare professional. After the plasma has been analyzed, step (308) proceeds to a step (310).

In step (310), the data collected in step (308) regarding the plasma sample is displayed on a display screen to the healthcare professional. The display screen of method (300) may be similar to display screen (134) as discussed above. Thereafter, step (310) moves to a step (312), whereby the healthcare professional reviews the data and determines whether the blood sample is acceptable. If the blood sample is not acceptable, step (312) moves to a step (314), where the healthcare professional initiates further interaction with the patient. Thereafter, method (300) ends. If the blood sample is acceptable, step (312) moves to a step (316).

In step (316), a labeling system of holder prints information onto a label and affixes the label onto the tube. Labeling system and label are similar to labeling system (142) and label (122), respectively, as discussed above. The holder may acquire patient information or other data via a communication port connected to a medical records system and pass this information to the labeling system for printing of the label. In some versions of step (316), the labeling system receives patient data from the medical records system and prints this information onto the label. The label is then affixed to the tube to allow for handling and movement of the tube with proper identification. Thereafter, method (300) ends.

III. Exemplary Device for Blood Collection and Separation

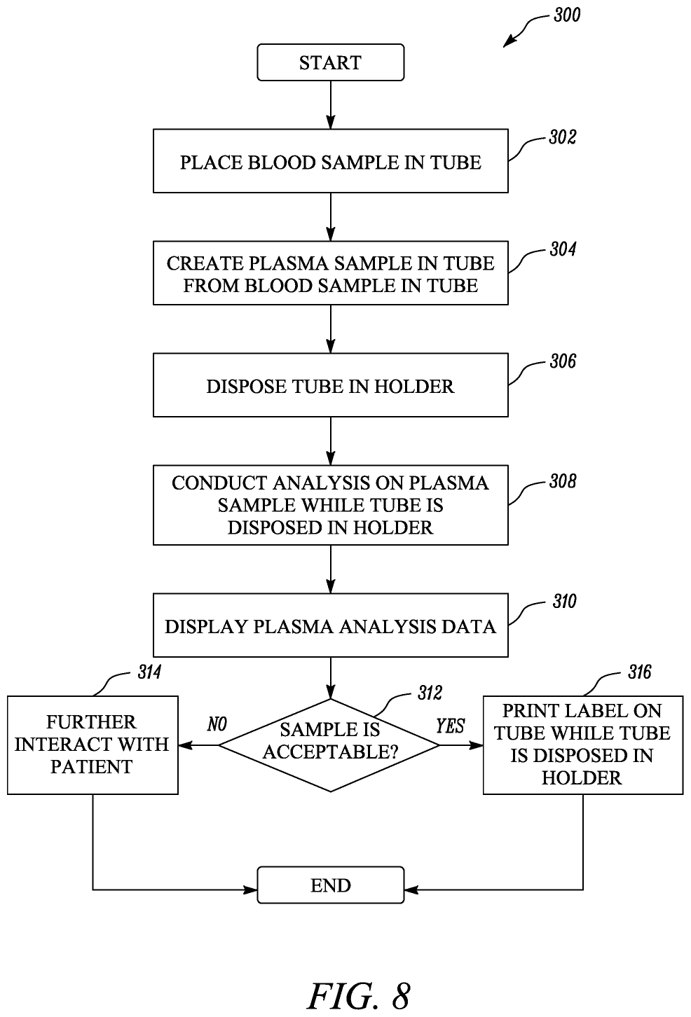

Referring now to FIG. 9, an exemplary device for blood collection and separation of the present disclosure is depicted as a device (401). Device (401) is configured to receive a needle and be inserted into the vein of a patient to draw a blood sample. Device (401) is configured to both draw and separate the blood sample. During blood draw, device (401) may separate the blood sample into separate materials such as whole blood, plasma, and/or serum and move these separated materials into isolated chambers. The blood separation technology may use different methods for separating sample types. For example, filters, anticoagulants, resins, micro-channels, special coatings or similar may be used. In some versions of device (401), one chamber may have an EDTA coating for whole blood separation, one chamber may have a plasma filter and EDTA for plasma separation, and one chamber may have spray silica coating for serum separation. Or one or more channels may include microfluidics or the like. Thereafter, device (401) may be loaded directly onto a rack of an analyzer instrument for processing. Alternatively, the individual containers of separated material may be removed from device (401) and loaded into a rack of the analyzer instrument. Portions of device (401) may include an identification element such as a bar code, color code, or RFID tag to provide information to the analyzer instrument.

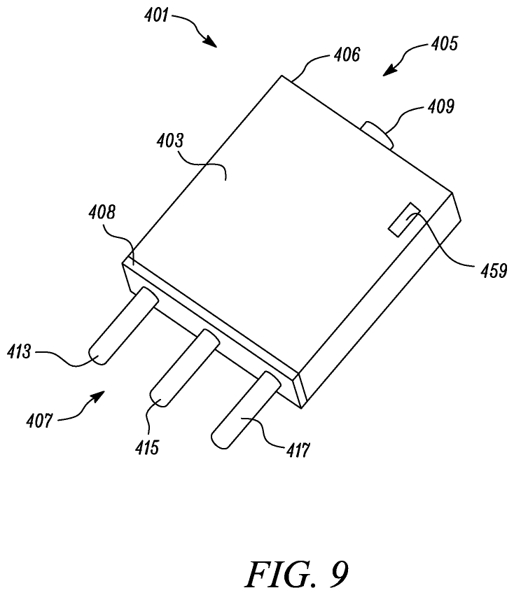

Device (401) includes a main body (403) extending from a front end (405) to a back end (407) and having a front wall (406) disposed at front end (405) and a back wall (408) disposed at back end (407). Device (401) includes a needle attachment feature (409) disposed at front end (405) and extending from front wall (406). Needle attachment feature (409) is configured to couple a needle element (411) (shown in FIG. 10) to main body (403). Further, needle element (411) is configured to transfer a blood sample from needle element (411) through front wall (406) and into main body (403). Needle element (411) may comprise a blood draw needle, a finger stick needle, or any other mechanism for drawing a blood sample from a patient.

As shown in FIGS. 9 and 10, multiple chambers are disposed at back end (407) of main body (403). In some versions of device (401), a first chamber (413), a second chamber (415), and a third chamber (417) are disposed at back end (407) of main body (403). In other versions of device (401), less than three or more than three chambers are disposed at back end (407) of main body (403). In some versions of device (401), one or more of first chamber (413), second chamber (415), and/or third chamber (417) are removably secured to back wall (408). In some versions of device (401), one or more of first chamber (413), second chamber (415), and/or third chamber (417) comprise a test tube.

In some versions of device (401), one or more of first chamber (413), second chamber (415), and/or third chamber (417) include an interior surface, wherein the interior surface is coated with a layer of silica. For example, as shown in FIG. 10, first chamber (413) includes an interior surface (419) coated at least in part with a layer of silica (421). In some versions of device (401), one or more of first chamber (413), second chamber (415), and/or third chamber (417) include an interior surface, wherein the interior surface is coated with an anticoagulant. For example, as shown in FIG. 10, second chamber (415) includes an interior surface (423) coated at least in part with a layer of anticoagulant (425).

As shown in FIG. 10, device (401) includes a separation element (427) disposed within main body (403). Separation element (427) is configured to separate a blood sample acquired through needle element (411) into separate portions and thereafter transfer these portions into the chambers disposed at back end (407) of main body (403). In some versions of device (401), separation element (427) is configured to separate a blood sample into a first portion (429), a second portion (431), and a third portion (433). Thereafter, device (401) is configured to transfer first portion (429) into first chamber (413), second portion (431) into second chamber (415), and third portion (433) into third chamber (417).

In some versions of device (401), separation element (427) is configured to separate a blood sample into a sample of whole blood and deposit the sample of whole blood into one of the chambers. In some versions of device (401), separation element (427) is configured to separate a blood sample into a sample of plasma and deposit the sample of plasma into one of the chambers. In some versions of device (401), separation element (427) is configured to separate a blood sample into a sample of serum and deposit the sample of plasma into one of the chambers. Separation element (427) may comprise or include a plasma separation system (435), a micro-channel (437), a filter (439), an anticoagulant, or a resin for use in separating a blood sample into different portions.

As shown in FIG. 6A, plasma separation system (435) may comprise a microfluidic separation system such as lateral cavity acoustic transducer system (113) having a plurality of segments, with one segment (115) of lateral cavity acoustic transducer system (113) depicted in FIG. 6B. In general, lateral cavity acoustic transducer system (113) receives as input whole blood and separates the whole blood into plasma and the remainder as the blood moves through the various segments. The relative movement of blood cells and other non-plasma elements is retarded or slowed to allow the plasma of the whole blood to be expelled from lateral cavity acoustic transducer system (113) first, thus separating the plasma from the whole blood for use as needed by separation element (427). Details of lateral cavity acoustic transducer system (113) and segment (115) have been discussed above with reference to FIGS. 6A and 6B. Exemplary lateral cavity acoustic transducer systems (213A) and (213B) have also been discussed above with reference to FIGS. 7A and 7B, respectively. In some examples, plasma separation system (435) may comprise lateral cavity acoustic transducer system (213B).

One or more of first chamber (413), second chamber (415), and/or third chamber (417) may include an identification element (441) such as a bar code, an RFID tag, or any other mechanism for identifying a specific chamber. Identification element (441) may be read by device (401) or another separate device for identifying a specific chamber. In some versions of device (401), identification element (441) is recognized by separation element (427) and separation element (427) is configured to separate the blood sample into a particular portion based at least in part on identification element (441). For example, first chamber (413) may include a particular identification element (441) associated with a request for a deposit of plasma therein. Separation element (427) recognizes the request for a deposit of plasma in first chamber (413) by identification element (441) and proceeds to separate a blood sample into a plasma portion and deposit this plasma portion into first chamber (413).

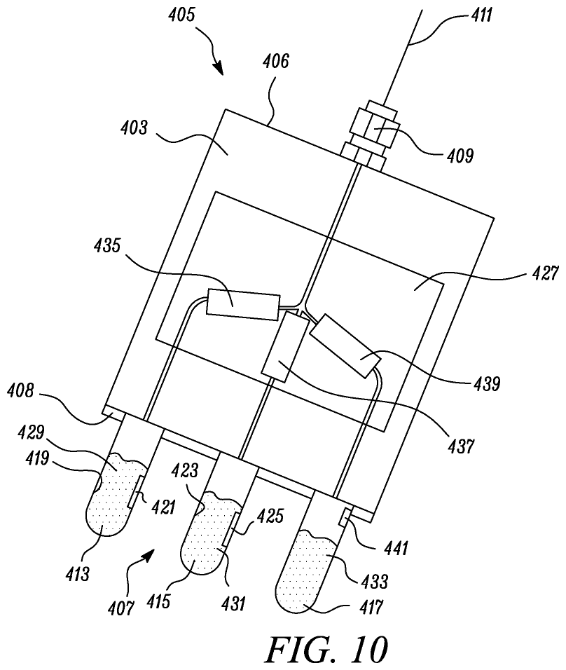

In some versions of device (401), one or more of first chamber (413), second chamber (415), and/or third chamber (417) are removably secured to back wall (408). Once the desired portions are separated out from a blood sample and transferred into first chamber (413), second chamber (415), and/or third chamber (417), the user may remove one or more of first chamber (413), second chamber (415), and/or third chamber (417) and deposit the removed chamber onto a rack (443) of an analyzer instrument (not shown), as shown in FIG. 11. Rack (443) or any other portion of the analyzer instrument may be configured to read any identification elements (441) of first chamber (413), second chamber (415), and/or third chamber (417) and to proceed with analyzing the particular chamber based on the associated identification element (441).

A vacuum is created within main body (403) to draw a blood sample from a patient once needle element (411) is connected to a patient's vein. As illustrated in FIG. 10, in some versions of device (401), needle attachment feature (409) cooperates with needle element (411) and main body (403) to utilize the internal vacuum when the user pushes main body (403) into needle element (411) and the blood is drawn automatically into main body (403).

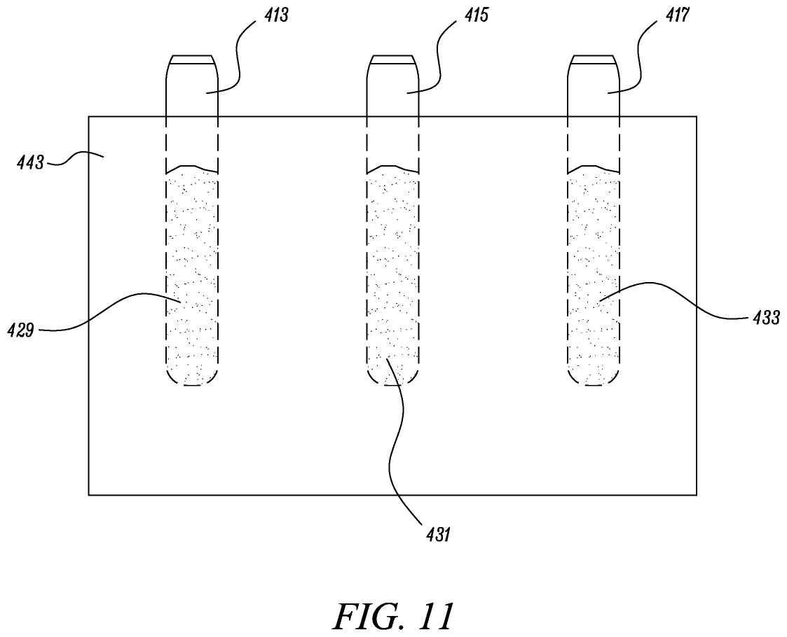

As illustrated in FIG. 12, in other versions of device (401), a syringe element (445) is coupled with first chamber (413), second chamber (415), and/or third chamber (417) and may be drawn away from back wall (408) to create the vacuum in main body (403). More specifically, syringe element (445) may include a handle (447), one or more stoppers (449), and a neck (451) extending between handle (447) and each stopper (449). The number of stoppers (449) correspond to the number of chambers. In the example illustrated in FIG. 12, three stoppers (449) are present, with first chamber (413), second chamber (415), and/or third chamber (417) each having one of stoppers (449) disposed therein. The user disposes each stopper (449) in the corresponding first chamber (413), second chamber (415), and/or third chamber (417) and when needle element (411) is connected to a patient's vein, pulls handle (447) in the direction of Arrow D (FIG. 12) to create a vacuum in main body (403) and draw a blood sample into separation element (427).

Once the user is done drawing a blood sample into main body (403), the user may disconnect and discard a disposable portion (452) of syringe element (445) to decrease the overall profile or size of device (401). To that end, some versions of syringe element (445) include a perforation or other type of breakaway feature on each neck (451) to facilitate removable of disposable portion (452), which includes handle (447) and at least a portion of neck (451). As shown in FIG. 12, a perforation (453) is provided on each neck (451). Once a blood sample is sufficiently drawn into main body (403), device (401) is removed from the patient. Thereafter, the user manipulates handle (447) to snap off disposable portion (452) from the remainder of syringe element (445) at each perforation (453). Each stopper (449) remains within one of first chamber (413), second chamber (415), and/or third chamber (417) to continue to cap the fluid within each chamber (413, 415, 417). The user may then discard disposable portion (452).

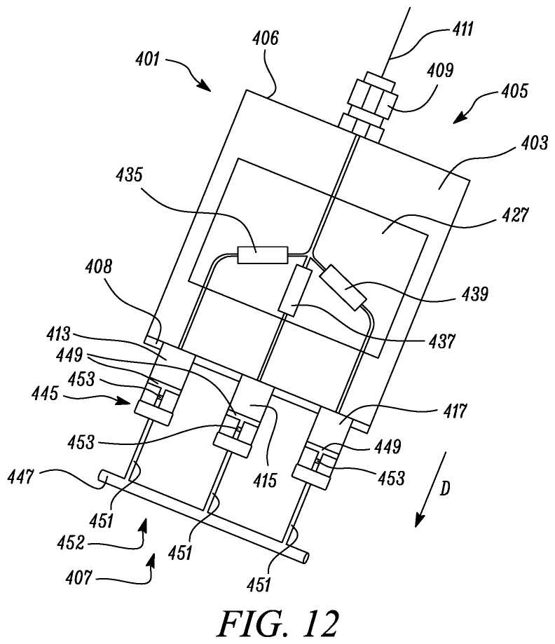

As illustrated in FIG. 13, in some versions of device (401), the user may omit removing first chamber (413), second chamber (415), and/or third chamber (417) from main body (403) and may place main body (403) directly into a device receptacle (455) of a rack (457) of an analyzer instrument (not shown). Rack (457) or any other part of analyzer instrument may be configured to detect the receipt of main body (403) therein and proceed with analyzing the material within first chamber (413), second chamber (415), and/or third chamber (417) accordingly. Main body (403) may include an identification element (459) comprising a bar code, an RFID tag, or any other mechanism for identifying a specific main body (403). Rack (457) may include a corresponding reader (461) for reading identification element (459) and using the information contained therein to determine how to analyze or process the material within first chamber (413), second chamber (415), and/or third chamber (417).

IV. Exemplary Methods for Blood Collection and Separation

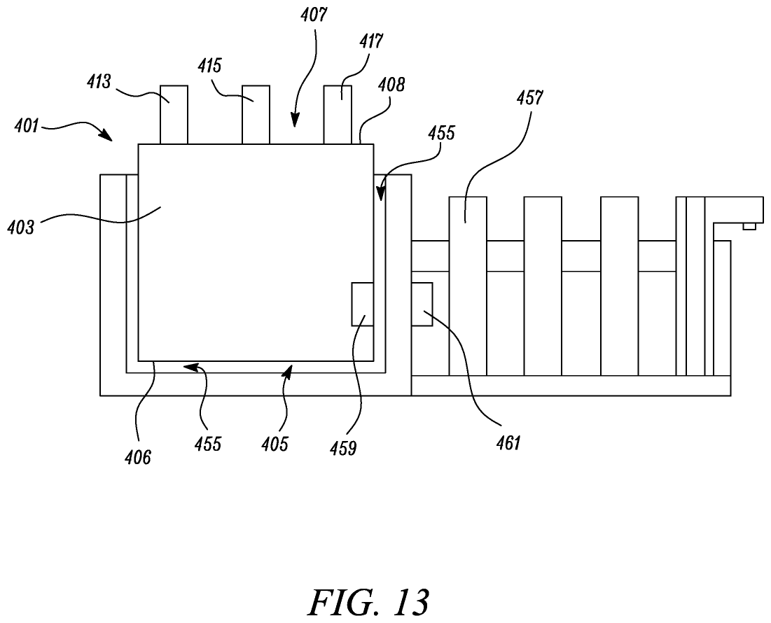

As shown in FIG. 14, various methods may be employed for blood collection and separation using a device similar to device (401) as described above. An exemplary method (501) begins with a step (503), whereby a needle element similar to needle element (411) is attached to a device similar to device (401) and inserted into a patient. Thereafter, step (503) proceeds to a step (505). In step (505), a vacuum is created in the device. The creation of a vacuum may be via any method common in the art. For example, a vacuum may be created by pressing on the device to expel air through a one-way valve. In another example, a syringe element similar to syringe element (445) may be incorporated into the device, whereby pulling on the syringe element creates a vacuum in the device. Once a vacuum is created in the device, step (505) moves to a step (507).

In step (507), a sample of blood is drawn from the patient through the needle element and into a main body of the device. Therein, the blood sample is separated into different portions. In some versions of step (507), the blood sample may be separated by a separation element similar to separation element (427). More specifically, in some versions of step (507), the blood sample may be separated into a whole blood portion, a plasma portion, and a serum portion and moved into isolated chambers such as first chamber (413), second chamber (415), and/or third chamber (417) as described above.

The mechanism for creating the plasma portion may be in the form of a microfluidic separation system such as a lateral cavity acoustic transducer system or a similar system for creating the plasma portion within the separation element. Lateral cavity acoustic transducer system may apply an acoustic energy to a main channel and associated lateral cavities to retard the movement of cells within the whole blood, leaving the plasma to exit the lateral cavity acoustic transducer system first, thus producing plasma from whole blood without centrifugation.

Thereafter, step (507) moves to a step (509). In step (509), the separated portions of the blood sample are loaded into a rack similar to either rack (443) or rack (457) above. In some versions of step (509), the portions are removed from the main body of the device and loaded directly into the rack. In other versions of step (509), the portions remain connected with the main body of the device and the main body is loaded into the rack for further processing. Once the separated portions of the blood sample are loaded into the rack, method (501) proceeds to end.

V. Exemplary Analyzer

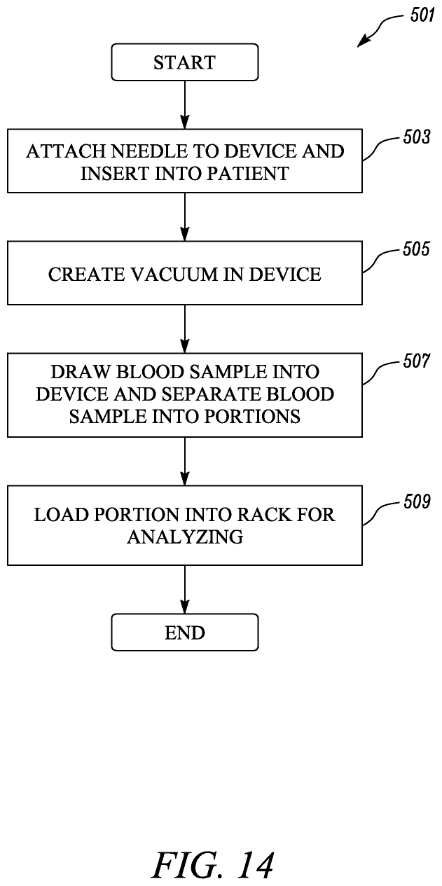

An exemplary analyzer (601) of the present disclosure is depicted in FIG. 15. Analyzer (601) is configured to receive a tube (603) containing a whole blood sample (605) therein. Analyzer (601) includes a separation system (607), a movement system (609), and an analysis system (611). Separation system (607) may be used to separate a whole blood sample into a separated sample. In some versions of analyzer (601), the separated sample may comprise either a plasma sample or a serum sample. Analysis system (611) is configured to conduct testing and analysis on the separated sample deposited therein. Analysis system (611) may comprise a chemistry analyzer, an immunoassay analyzer, a molecular analyzer, and/or a mass spectrometry analyzer.

Separation system (607) is configured to separate all or a portion of whole blood sample (605) into a separated sample such as a plasma sample or a serum sample. Separation system (607) may utilize or comprise any mechanism for converting whole blood sample (605) into a separated sample. For example, separation system (607) may include a microfluidic separation system. Some versions of the microfluidic separation system may comprise a lateral cavity acoustic transducer (LCAT) system or otherwise use LCAT technology therein.

Analyzer (601) may be combined with a cassette (617) to form an analyzer system (602). As such, some versions of separation system (607) include a main body (613) which defines a cassette receptacle (615). Cassette receptacle (615) is configured to removably receive cassette (617) therein.

Cassette (617) may be a consumable and disposable component of separation system (607) containing the mechanism for converting whole blood sample (605) into the separated sample. For example, cassette (617) may contain the above-mentioned elements relating to a microfluidic separation system and/or LCAT technology, allowing these mechanisms to be discarded after coming into contact with whole blood sample (605). By providing a disposable separation component such as cassette (617), the cleaning and sterilizing required when using blood-based diagnostic equipment is reduced. Cassette (617) may provide for multiple uses before being discarded.

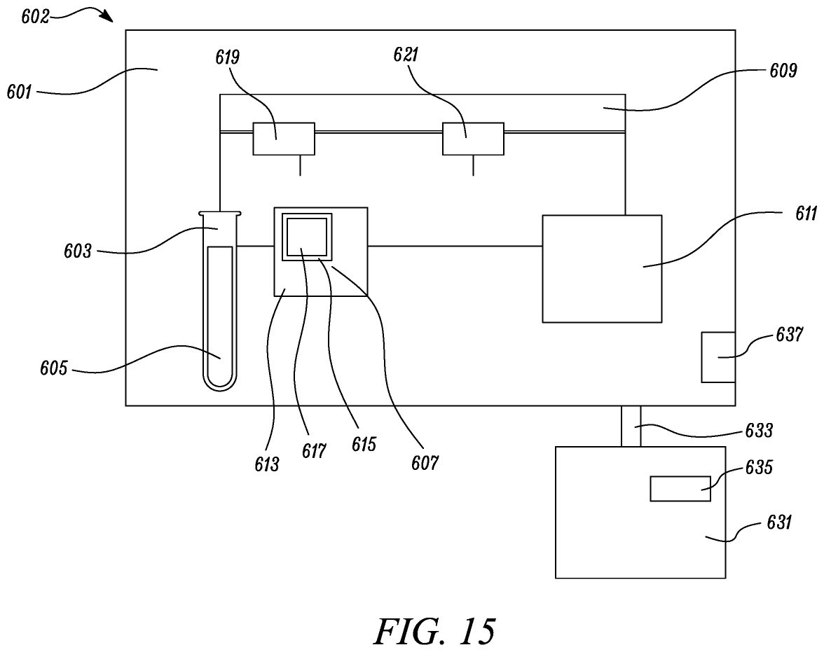

An exemplary cassette (617) is shown in greater detail in FIG. 16. Cassette (617) is configured to be removably received within cassette receptacle (615) of separation system (607). Some versions of cassette (617) are configured to receive all or a portion of whole blood sample (605) therein and create a separated sample, being either a plasma sample or a serum sample, from whole blood sample (605) without centrifugation. Some versions of cassette (617) include one or more separation channels (623). Each separation channel (623) extends from a first portion (625) to a second portion (627) to a third portion (629). In general, the portion of whole blood sample (605) is deposited in first portion (625) and travels into second portion (627) where a separated sample is generated therefrom. The separated sample thereafter travels into third portion (629) for further use by analyzer (601). While multiple separation channels (623) are shown in FIG. 16, cassette (617) may alternatively include one separation channel (623) per cassette (617). Alternatively, each separation channel (623) may produce multiple separated samples from whole blood sample (605). In some versions of separation channel (623), this is accomplished by having more than one third portion (629) per separation channel (623).

First portion (625) and third portion (629) may comprise wells such as those wells found in assay or other microbial instruments. First portion (625) and third portion (629) may include a film or other covering over unused wells in cassette (617). Third portion (629) may be an open or otherwise uncovered well. Separation system (607) is configured to puncture the film or covering of first portion (625) in order to open up the well for receiving the whole blood sample (605). Similarly, separation system (607) is configured to puncture the film or covering of third portion (629) in order to open up the well for removing the separated sample formed via the particular separation channel (623). As shown in FIG. 16, first portion (625A) and third portion (629A) are depicted as punctured and thus the associated separation channel (623) has been used by analyzer (601). Analyzer (601) will move to the next unused separation channel (623) when creation of a new separation sample is desired from either whole blood sample (605) or a different batch of whole blood. In this way, cassette (617) may be used to facilitate non-centrifugal separation of whole blood numerous times before being needing replaced and further eliminates the need for cleaning or sterilizing of separation equipment.

Second portion (627) generates a separated sample from the portion of whole blood sample (605) inserted into first portion (625) without centrifugation. Second portion (627) may include a microfluidic separation system such as an LCAT system and may be configured to separate whole blood sample (605) into a plasma sample and/or a serum sample.

Movement system (609) is configured to transfer at least a portion of whole blood sample (605) from tube (603) to separation system (607). Movement system (609) includes a first transfer element (619) movable between tube (603) and separation system (607). First transfer element (619) is configured to obtain all or a portion of whole blood sample (605) from tube (603) and deposit it into separation system (607). First transfer element (619) may include a pipette or any other element or mechanism for drawing and depositing all or a portion of whole blood sample (605).

In some versions of analyzer (601), movement system (609) is configured to transfer at least a portion of the separated sample generated by separation system (607) from separation system (67) to analysis system (611). In some versions of analyzer (601), movement system (609) includes a second transfer element (621) movable between separation system (607) and analysis system (611). Second transfer element (621) is configured to obtain at least a portion of the separated sample from separation system (607) and deposit it into analysis system (611).

In other versions of analyzer (601), tube (603) may be placed directly onto an adapter (not shown) which draws the portion of whole blood sample (605) into separation system (607), without the need for first transfer element (619). Similarly, in some versions of analyzer (601), third portion (629) may provide the separated sample directly to analysis system (611) through features such as a cuvette or similar element, without the need for second transfer element (621).

VI. Exemplary Plasma Separation

Generating plasma without the use of centrifugation in conjunction and connection with analyzer (601) or any other biochemical analyzer which may require plasma samples generated from whole blood before the actual diagnostic analysis has numerous advantages. Currently, centrifugation creates a bottleneck in the laboratory workflow. Blood samples that need to be spun to generate plasma can take up to five to fifteen minutes onboard the centrifuge in order to generate a usable plasma. Further, once the centrifugation starts the laboratory technician is forced to wait until this batch is complete. This workflow causes delays and potentially leads to a delay of the results.

The common laboratory workflow requires the laboratory technician to perform various steps and can potentially be erroneous before loading the spun plasma sample onboard the instrument. From entry into the lab, this workflow involves: (a) accessioning samples; (b) sorting samples; (c) labeling samples; (d) racking samples; (e) centrifuge samples; (f) de-capping samples; (g) carrying samples to required workstations; (h) placing samples onboard instrument; (i) removing samples from instrument; (j) recapping samples; and (k) storing samples.

Centrifugation is currently the main method for generating plasma from blood. As detailed above, centrifugation takes at least five to fifteen minutes to generate plasma and requires several manual steps from the user. Centrifugation also requires batching the samples. Further, the centrifugation process may lead to sample hemolysis.

A. Plasma Separation Using Lateral Cavity Acoustic Transducer

In some versions of separation system (607), plasma may be generated from whole blood using a microfluidic separation system such as an LCAT system. LCAT technology may be implemented into a consumable (i.e., disposable) element which is used in a specific device that can be directly or indirectly connected to analyzer (601) or another biochemical analyzer. In some versions, cassette (617) includes LCAT technology. In other versions, cassette (617) is replaced by using individual consumable elements such as a single use consumable using LCAT technology or a grouping of LCAT consumable elements which can produce plasma from multiple whole blood samples within the same consumable unit.

In general, LCAT utilizes waves of a specific frequency which may be produced via a transducer or “LCAT chip” to create structure known as vortices to delay the flow of larger particle within the blood and allow the plasma portion to move ahead of the cell portion of blood. The plasma separation module provides the capability for several of said consumable elements incorporating LCAT structures to be processed simultaneously or in parallel to one another and generate sufficient plasma volume for diagnostic testing such as in analyzer (601).

Some versions of the plasma generation module associated with separation system (607) use an array of piezoelectric transducers as a wave generator and couples the consumable elements to the wave generators with the help of gel pads. The module is intended to reduce the number of manual steps required currently to generate plasma and process samples with the help of a specific device which enables parallel processing. This configuration provides a seamless transition from loading the whole blood sample into analyzer (601) and/or separation system (607), where plasma would be generated. The new plasma generated would automatically be presented to a sample probe, movement system (609), or similar of analyzer (601) for sampling without any more input from the user.

More specifically, as shown in FIG. 6A, separation system (607) may comprise a microfluidic separation system such as a lateral cavity acoustic transducer system (113) having a plurality of segments, with one segment (115) of lateral cavity acoustic transducer system (113) depicted in FIG. 6B. In general, lateral cavity acoustic transducer system (113) receives as input whole blood and separates the whole blood into plasma and the remainder as the blood moves through the various segments. The relative movement of blood cells and other non-plasma elements is retarded or slowed to allow the plasma of the whole blood to be expelled from lateral cavity acoustic transducer system (113) first, thus separating the plasma from the whole blood for use as needed by analyzer (601). Details of lateral cavity acoustic transducer system (113) and segment (115) have been discussed above with reference to FIGS. 6A and 6B. Exemplary lateral cavity acoustic transducer systems (213A) and (213B) have also been discussed above with reference to FIGS. 7A and 7B, respectively.

B. Stand-Alone Element

Some versions of analyzer (601) may be coupled with a stand-alone element (631) which may be separate from analyzer (601) or connected via a connection (633). Stand-alone element (631) encompasses an automated processing device which is able to process a consumable (635) of a specific size and generate plasma from whole blood while minimizing the manual interaction by the operator. Some versions of consumable (635) may incorporate LCAT features, such as those depicted in FIGS. 6A, 6B, 7A and 7B.

Stand-alone element (631) may process capped sample tubes by removing or penetrating the cap of the sample tube and drawing a defined volume of whole blood from the tube. Stand-alone element (631) may be fitted with a de-capping mechanism. This mechanism is able to automatically remove caps from the sample tubes and discard the removed caps into a waste chute. Alternatively, stand-alone element (631) may be fitted with a cap piercing probe. The probe may be configured to directly pierce the cap of a blood tube and directly draw the blood, without the need to de-cap. Either syringe pump technology or pressure/vacuum driving pump technology may be used to draw the whole blood into the probe. The probe may be motor driven and capable of moving along all three axes (XYZ) and may undergo a wash cycle with an appropriate reagent after each sampling to minimize the likelihood of sample contamination. All of the aforementioned movement or transfer steps may be provided through movement system (609) or any other system within analyzer (601) as discussed above.

Once drawn, stand-alone element (631) dispenses the whole blood into sample reservoir of consumable (635). Thereafter, plasma is generated via consumable (635). In some versions of consumable (635), plasma is generated using the LCAT principle and associated features, such as one or more features depicted in lateral cavity acoustic transducer system (113).

Once plasma is generated via consumable (635), the plasma is presented in one or several outlet reservoirs. In some versions of consumable (635), plasma is presented in several reservoirs to be able to generate aliquoting from the same sample if this is required by the operator or the underlying plasma testing. In some versions of stand-alone element (631), the outlet reservoir(s) of consumable (635) may be moved to an exposed position where it can be accessed by analyzer (601) for further use therein. Within stand-alone element (631), the whole blood samples and consumable (635) may be moved around by a sample transport module capable such as movement system (609) of accessing and moving both the sample tube and consumable (635). This can be accomplished by using a gantry system capable of moving along all three axes (XYZ). The gantry system can either make use of stepper motors in combination with a lead screw or a belt system, or it can also be powered by using a linear actuator. The sample transport module can also make use of a solenoid or an electromagnetic component to engage the sample tube and/or consumable (635). All of the aforementioned movement or transfer steps may be provided through movement system (609) or any other system within analyzer (601) as discussed above.

In those versions of consumable (635) utilizing the LCAT principle and associate features, once consumable (635) has been moved into a dedicated processing or buffer area, it will undergo plasma separation and this process will be automated by mechanically engaging a piezoelectric transducer such as transducer (123) and associated gel to an LCAT chip. This can be accomplished by attaching the transducer to a fixture mounted along a gantry system able to move along all three axes (XYZ). Consumables (635) and the interfacing transducers can be mounted in a horizontal or a vertical orientation to accommodate for space and throughput constraints of the separation system (607).

C. Integrated Handler Element

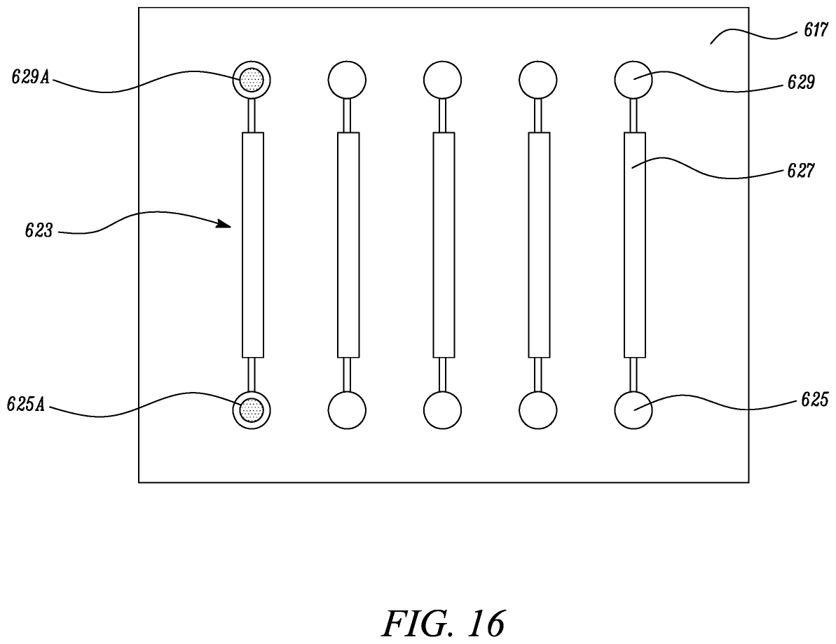

As shown in FIGS. 15 and 17A-17B, in those versions of analyzer (601) with a handler element for receiving racks of samples, an integrated handler element (637) with plasma separation features may be provided with features used to facilitate plasma generation. For example, handler element (637) may utilize LCAT technology and thus include a transducer such as transducer (123), as shown in FIG. 6B. Various features used in LCAT technology may be placed above or in space between the rack tracking system of analyzer (601) or other analyzers and the offloading area. For example, separation system (607) may be placed above or in the space between the current rack tracking system and the offloading area. Corresponding software may be coupled with analyzer (601) to create a separate plasma sample type that includes specific parameters to allow differentiation of serum and plasma within the existing rack system.

As shown in FIGS. 17A and 17B, integrated handler element (637) may include a rack loading station for plasma samples only, shown in FIGS. 16A and 16B as a rack loader (639), with a dedicated barcode reader (not shown) associated with the plasma loading station. Integrated handler element (637) may include a first sampling area (641), a second sampling area (643), a rack buffer area (645), and a decapper (647). In some versions of integrated handler element (637), workflow proceeds such that a technician loads a rack (640) of whole blood sample tubes into rack loader (639), shown as rack (640A). Thereafter, the rack is moved to sampling area (641), shown as rack (640B), whereby the decapper (647) removes the cap of the sample tubes. Thereafter a portion of the whole blood sample for a particular tube may be placed into a consumable or otherwise processed to initialize the plasma generation process. In some versions of integrated handler element (637), the portion of whole blood is placed into a consumable associated with another rack and this rack is moved into the rack buffer area, shown as rack (640C). For those consumables using LCAT technology, while in the rack buffer area transducer (632) may be employed to generate plasma. Thereafter, once plasma is generated from a particular sample, the rack and/or sample is moved to sampling area (643), shown as rack (640D), for acquiring the plasma sample. In some versions of integrated handler element (637), upon starting analyzer (601), the separation system (607) would immediately start processing the plasma from the whole blood without waiting for the rest of the sub-systems of analyzer (601) to initialize. All of the aforementioned movement or transfer steps may be provided through movement system (609) or any other system within analyzer (601) as discussed above.

Integrated handler element (637) may include a large volume whole blood sample probe capable of picking up 300 μL or more. The probe may utilize disposable pipette tips that can be pre-loaded into analyzer (601). In some versions of analyzer (601), these disposable pipette tips are disposed under the rack loader elements. In some other versions of integrated handler element (637), no disposable tips are used and separation system (607) includes a wash station for washing and sterilizing the probe. In still other versions of integrated handler element (637), the standard sample probe may be used to withdraw plasma directly from the LCAT elements such as an LCAT collection cup. With respect to previous handler elements, integrated handler element (637) may include altered dimensions to increase collection capacity to 100 μL or more and may also have side cup feature added to collect plasma vertically.

Integrated handler element (637) may include updates or configuration regarding the placement, movement, and disposal of consumables such as an LCAT element. For example, horizontal or vertical pre-loaded blocks or rolls of individual or sheets of LCAT elements may be stacked and pushed into position with respect to the sample probes of the separation system (607) for whole blood dispensing. Thereafter, these LCAT elements may be moved across a series of transducer plates and into position for the main sample probe of analyzer (601). A queuing mechanism may be provided for those consumables and/or LCAT elements with plasma awaiting sampling to prevent backlog within analyzer (601).

In another embodiment, multiple LCAT elements may be imprinted onto a continuous polymer LCAT sheet which is then rolled into a compact spool. After mounting on a spindle, the LCAT sheet and spool is unwound, exposing LCAT elements for use as desired or needed. After the plasma has been generated and removed for testing, another spindle winds the LCAT sheet and the used LCAT elements into a new spool, while simultaneously exposing unused LCAT elements on the LCAT sheet for use.

Separation system (607) may include various timing considerations for maximizing efficient throughput from separating plasma, delivering plasma, and cleaning the plasma probe. For example, as consumables move from initial fill with whole blood to plasma sampling by the sample probe, this movement may be timed to allow for reagent and sample dispensing according to each underlying assay's settings. The placement of the consumable into final position under the sample probe can be the trigger for reagent dispensing for cleaning of the probe.

D. Integrated Wheel Element

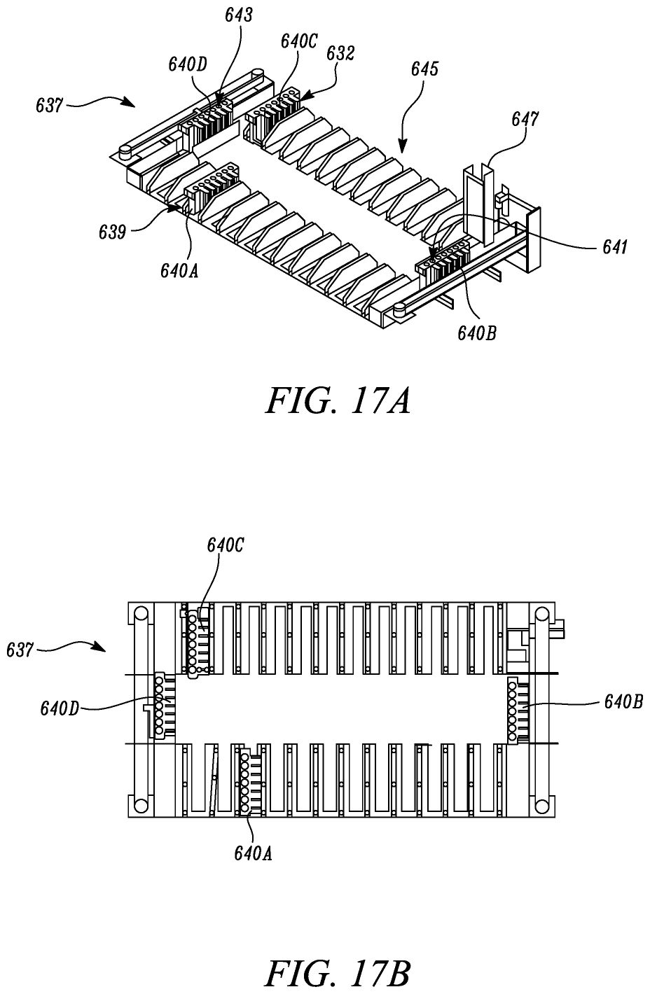

As shown in FIGS. 18A, 18B, and 18C, LCAT technology and/or LCAT elements may be an integrated component of a reaction wheel (649) of analyzer (601). In some versions of analyzer (601), a reusable LCAT consumable (651) is provided within a holder (653) of reaction wheel (649) and may generate enough plasma to run the test directly in a cuvette (655) of reaction wheel (649). Reusable LCAT consumable (651) may be a reusable consumable that undergoes the same wash cycle as cuvette (655).

In these versions of analyzer (601), a tube containing a whole blood sample is loaded manually by the lab technician into a common rack. The common rack would be carried over to analyzer (601) and loaded into the sample loader space. The barcode in each sample will be read by the onboard barcode reader. In some versions of analyzer (601), a decapper is available and thus the sample tube would first be decapped onboard. In other versions of analyzer (601) a cap piercing probe is available and thus the next step will be performed by that probe. If neither a decapper nor a cap piercing probe is available, a user removes the cap.

An amount of whole blood from the sample is then aspirated to generate enough plasma for the specified test. The whole blood is thereafter delivered to reusable LCAT consumable (651) onboard reaction wheel (649) by a sample probe. In some versions of analyzer (601), the sample probe is capable of aspirating a minimum of 120 μL.

The plasma probe would subsequently be cleaned accordingly with the appropriate chemicals to minimize the chance of cross contamination of the next sample. Reaction wheel (649) includes a transducer (657) similar to transducer (123) as described above. Once the lifecycle of the reusable LCAT consumable (651) is complete, the LCAT consumable (651) is ejected from a slot in reaction wheel (649) into a trash chute, and holder (653) is loaded with another reusable LCAT consumable (651). Analyzer (601) may also include a pump to deliver the correct volume of plasma to reaction wheel (649).

Once every tube in the rack has been sampled, that rack will go in the buffer area of the instrument until the results are released. Once the results are released, the rack is moved to the front loader where the customer can take the samples and spin them for storage. Reusable LCAT consumables (651) may be loaded into analyzer (601) as part of a cassette, which may be in the form of cassette (617). A user would load the cassette into analyzer (601) and refill as needed. All of the aforementioned movement or transfer steps may be provided through movement system (609) or any other system within analyzer (601) as discussed above.

E. Integrated Cap Element

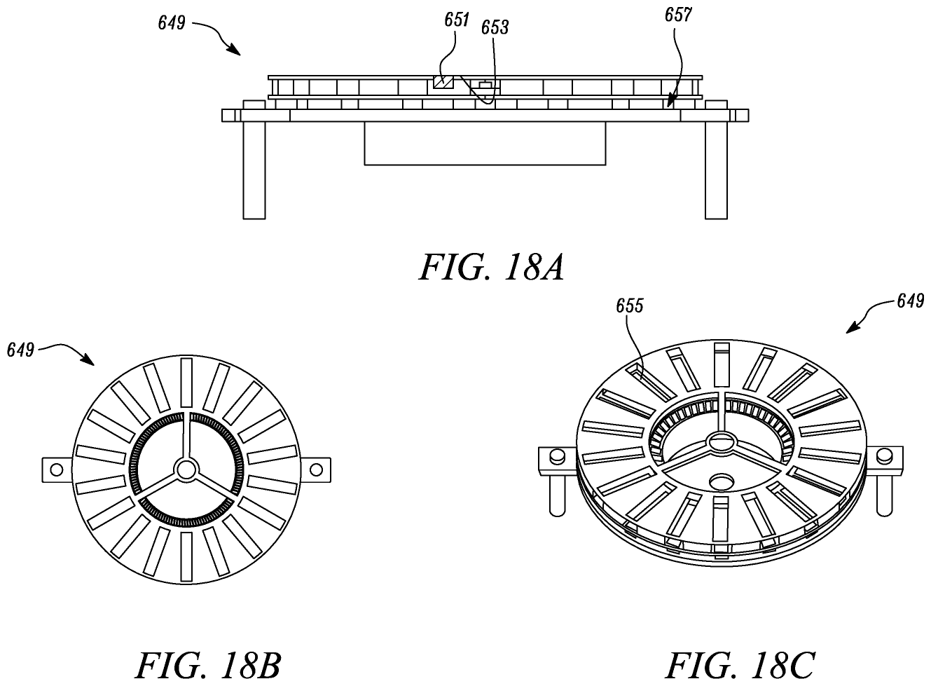

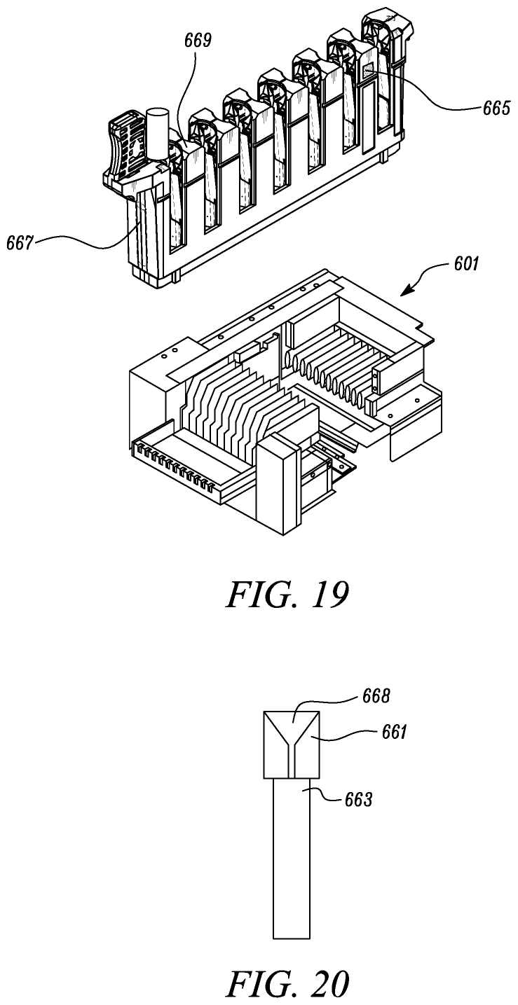

As shown in FIGS. 19 and 20, some versions of analyzer (601) may include features configured to retrieve plasma from the blood collection tubes placed therein. In these versions of analyzer (601), an LCAT insert (661) is connected to a blood collection tube (663). Once the whole blood is collected in blood collection tube (663), blood collection tube (663) is capped with LCAT insert (661). LCAT insert (661) processes a portion of the whole blood inside blood collection tube (663) and converts this portion of the whole blood into plasma using LCAT methods. The plasma is thereafter accumulated in the top of LCAT insert (661) in a reservoir (668) where it is available for a probe of analyzer (601). This probe retrieves an amount of the plasma from reservoir (668) of LCAT insert (661) and used it within analyzer (601) as needed.

In some versions of LCAT insert (661), a transducer (665) similar to transducer (123) is disposed on a rack (667) and configured to receive blood collection tube (663) with LCAT insert (661) thereon. Transducer (665) may be an air transducer or include various features relating to LCAT technology such as a gel disposed between transducer (665) and potential blood collection tube (663) within rack (667). A locking mechanism (669) may be provided to ensure proper contact is being made between blood collection tube (663) and/or LCAT insert (661) and rack (667) and/or transducer (665) to ensure proper separation of the plasma. LCAT insert (661) may include a consumable that is replaceable by the user as needed.

In operation, blood collection tube (663) with LCAT insert (661) thereon is loaded into rack (667) and carried to analyzer (601) by a user. Rack (667) is thereafter loaded onboard analyzer (601) and moved to a buffer area where the whole blood inside blood collection tube (663) with LCAT insert (661) thereon undergoes plasma separation through agitation by transducer (665). Once plasma generation is been completed and an amount of plasma is accumulated within reservoir (668), analyzer (601) would move the appropriate rack to get sampled by the sampling probe and deliver the specified volume of plasma to the reaction cuvette of analyzer (601) and the sample probe would be washed accordingly. All of the aforementioned movement or transfer steps may be provided through movement system (609) or any other system within analyzer (601) as discussed above.

F. Cassette Element

In some versions of analyzer (601), cassette (617) may incorporate LCAT technology therein. For example, first portion (625), second portion (627), and/or third portion (629) of each separation channel (623) may include a microfluidic separation system such as an LCAT system and may be configured to separate whole blood sample (605) into a plasma sample and/or a serum sample. More specifically, first portion (625), second portion (627), and/or third portion (629) may comprise all or a portion of main channel (127), the plurality of lateral cavities (121), and/or transducer (123) configured to apply an external source of acoustic energy to second portion (627) and oscillate fluid within lateral cavities (121) of second portion (627).

G. LCAT Rack with External Transducer and Output Well

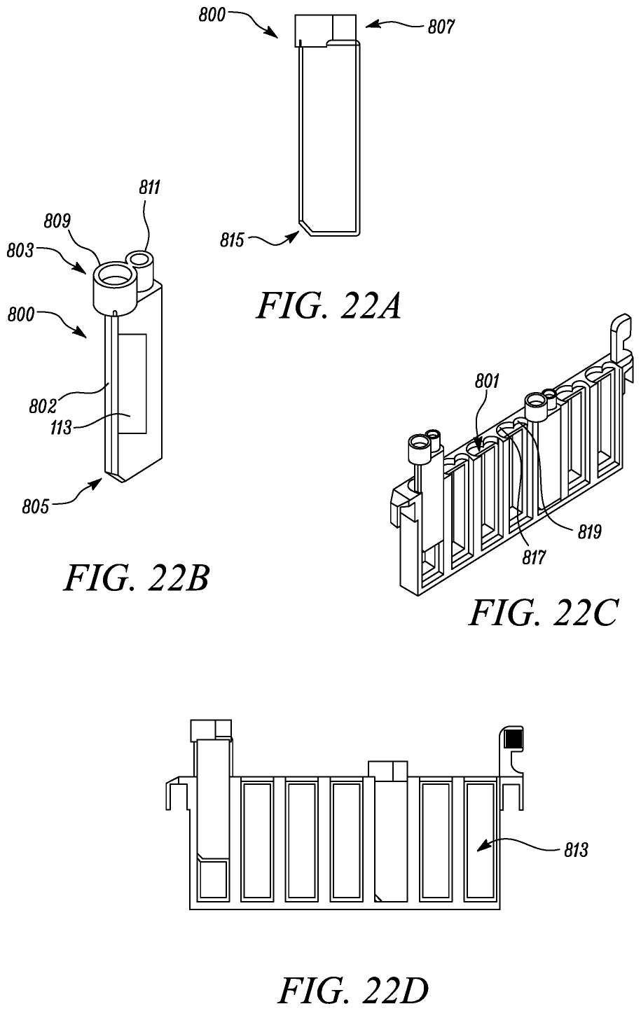

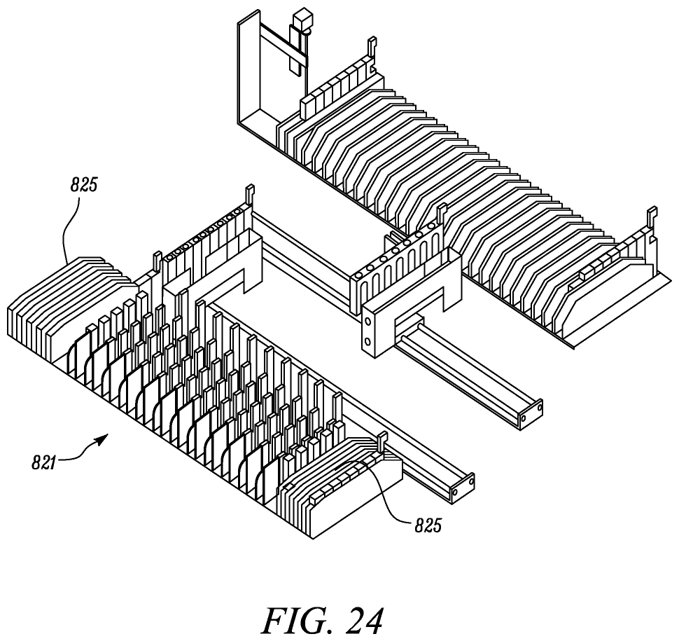

As shown in FIGS. 22A-22D, 23A-23B, 24, and 25A-25B, an LCAT chip (800) is provided along with an LCAT rack (801) and generally used to convert whole blood into plasma for further use within analyzer (601) by applying an external transducer (123) to LCAT rack (801) to oscillate each of the LCAT chips (800) disposed in LCAT rack (801) simultaneously.

In some versions of LCAT chip (800), one or more elements of lateral cavity acoustic transducer system (113) (“LCAT system”) is disposed within a housing (802) and used to convert whole blood into plasma within housing (802). For example, multiple segments such as segment (115) may be disposed within LCAT chip (800).

LCAT chip (800) extends from a top (803) to a bottom (805), with a well area (807) disposed at top (803). Well area (807) includes an input well (809) configured to receive whole blood therein. Input well (809) is operatively connected to LCAT system (113) disposed within housing (802) of LCAT chip (800), whereby the whole blood sample deposited in input well (809) is transferred through LCAT system (113) to generate plasma. While input well (809) is depicted as a cylindrical element in FIGS. 22A-22D, any orientation or shape of input well (809) may be used to facilitate the receipt of whole blood therein.

Well area (807) further includes an output well (811) configured to collect the plasma generated by LCAT system (113) within housing (802) and expose the plasma for further use as needed within analyzer (601). The plasma exposed within output well (811) may be collected by plasma sampling probe or similar sampling element and delivered to a reaction cuvette of analyzer (601) or any other desired element within analyzer (601). While output well (811) is depicted as a cylindrical element in FIGS. 22A-22D, any orientation or shape of output well (811) may be used to facilitate the collection and exposure of plasma generated within housing (802).

In some versions of LCAT chip (800), housing (802) is generally a flat, rectangular shaped body to enable housing (802) to fit vertically within a corresponding and complementary shaped slot (813) of LCAT rack (801). A notch (815) is provided and defined by housing (802) to ensure proper insertion into slot (813) and orient input well (809) and output well (811) accordingly. A corresponding alignment feature is disposed (not shown) in the internal area defined by slot (813) to prevent LCAT chip (800) from being pressed fully into slot (813) when oriented incorrectly. Further, LCAT rack (801) defines an input well receiving space (817) and an output well receiving space (819). Both input well receiving space (817) and output well receiving space (819) are sized accordingly to receive their respective portions of well area (807) and ensure LCAT chip (800) is properly inserted into LCAT rack (801) in the correct orientation.

As described above, a transducer is used to apply an external source of acoustic energy and oscillate fluid within lateral cavities and main channel of LCAT system (113), such as those disposed within housing (802) of LCAT chip (800). The transducer may be a piezo-electric transducer or any other style of transducer or mechanism for oscillation and may be located anywhere within analyzer (601) or may be disposed within each individual LCAT chip (800).

In the version of LCAT chip (800) depicted in FIGS. 22A-22D, 23A-23B, 24, and 25A-25B, a plasma separation module (821) includes a plurality of transducer slots (823), each associated with a transducer (825) similar to transducer (123) described above. Transducer slots (823) are sized and configured to receive one LCAT rack (801) therein and provide an abutting relationship between the associated transducer(s) (825) and LCAT chips (800) within LCAT rack (801). Per configuration of analyzer (601), as needed, acoustic energy is applied to each LCAT chip (800) in LCAT rack (801) through transducers (825) to separate the plasma from the whole blood. Once plasma is produced, the LCAT rack (801) is moved from transducer slot (823) to stand alone area (827), which are a series of slots sized to hold an LCAT rack (801) on plasma separation module (821) and await further movement within analyzer (601) as needed. Standalone area (827) may be used to hold empty LCAT common racks (801) or LCAT common racks (801) filled with yet to be used LCAT chips (800). In some versions of analyzer (601) and separation system (607), a user may load LCAT chips (800) directly onto a portion of plasma separation module (821) for use in analyzer (601).

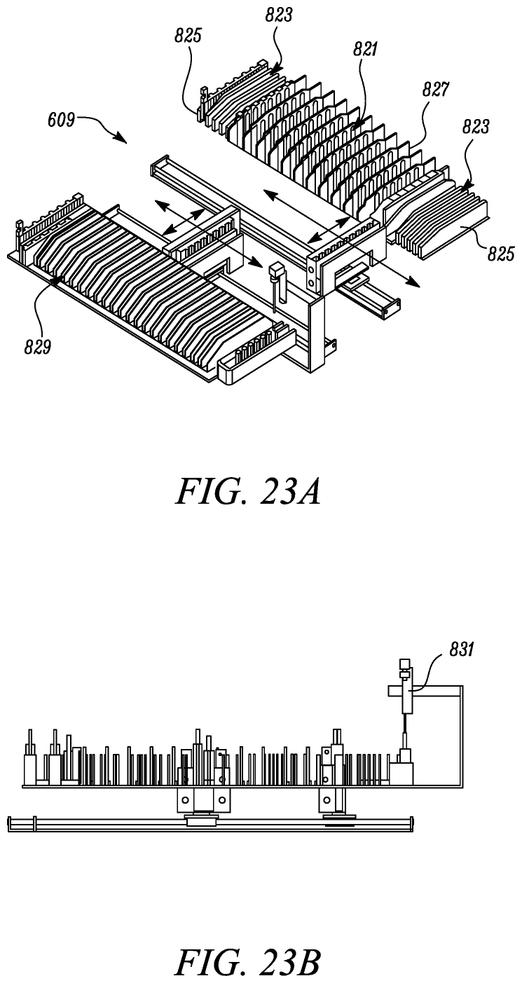

A gantry or movement element such as that described above with respect to movement system (609) may be used to collect whole blood from a particular tube in a sample loading area (829) and deposit the whole blood in a particular LCAT chip (800) within an LCAT rack (801). As shown in FIGS. 23A-23B, a sample probe (831) may be used to transfer whole blood to input wells (809) of LCAT chips (800).

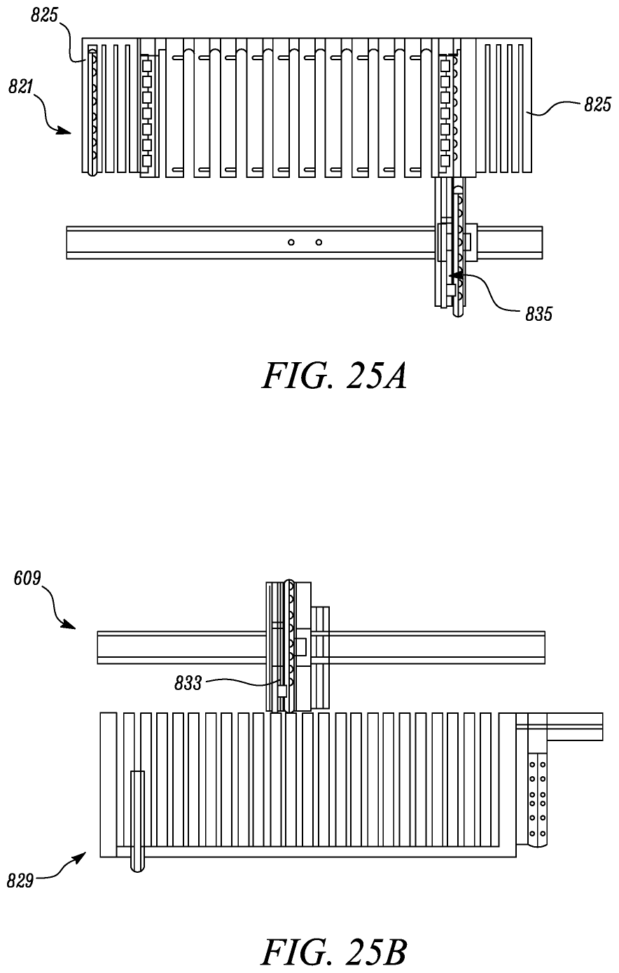

As shown in FIGS. 25A-25B, movement system (609) with respect to plasma separation module (821) may include two transport modules for moving blood sample racks and LCAT common racks (801) with respect to the above steps and procedures. For example, movement system (609) may include a dual rack transport module (833) for receiving a blood sample rack thereon and a LCAT rack (801) with at least one unused LCAT chip (800) therein. Sample probe (831) (FIGS. 23A-23B) draws a portion of the whole blood from the tub associated with the blood sample rack in dual rack transport module (833) and deposits it into input well (809) of an unused LCAT chip (800). Thereafter, LCAT rack (801) is moved into an LCAT rack transport module (835), which places the associated LCAT rack (801) into a transducer slot (823). Once the plasma is generated via transducer(s) (825), LCAT rack transport module (835) moves LCAT rack (801) into standalone area (827) to await further use by analyzer (601).

H. LCAT Rack with Internal Transducer and Dispense Tip

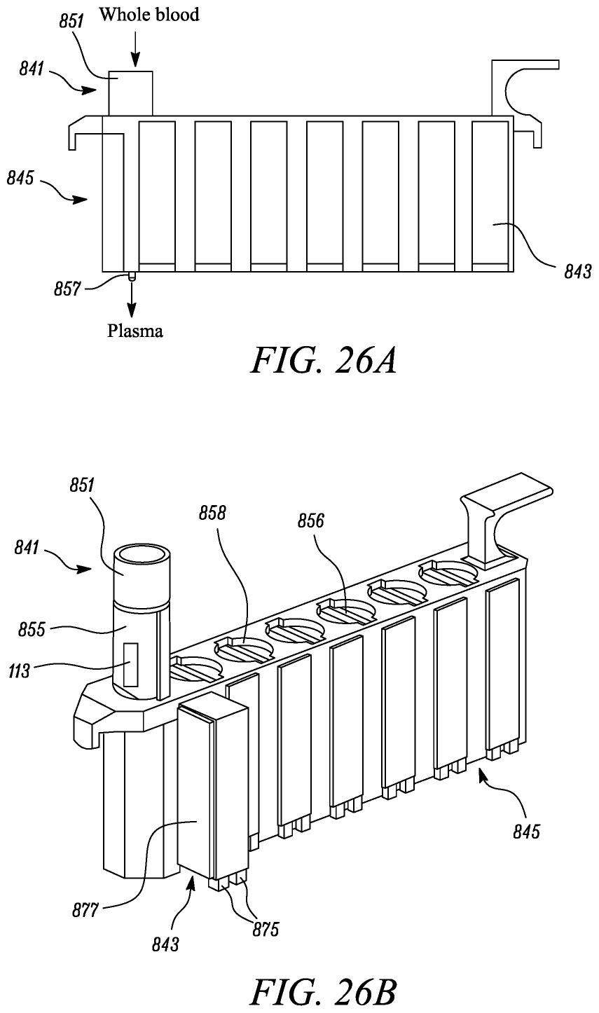

As shown in FIGS. 26A-26B, 27A-27D, 28A-28C and 29A-29C, an LCAT rack (845) may be provided and configured to receive both a plurality of LCAT chips (841) as well as a plurality of transducer cartridges (843). In general, each transducer cartridge (843) is associated with a corresponding LCAT chip (841) within LCAT rack (845) and is used to apply acoustic energy to the associated adjacent LCAT chip (841) as needed to separate the whole blood deposited into LCAT chip (841) into plasma.

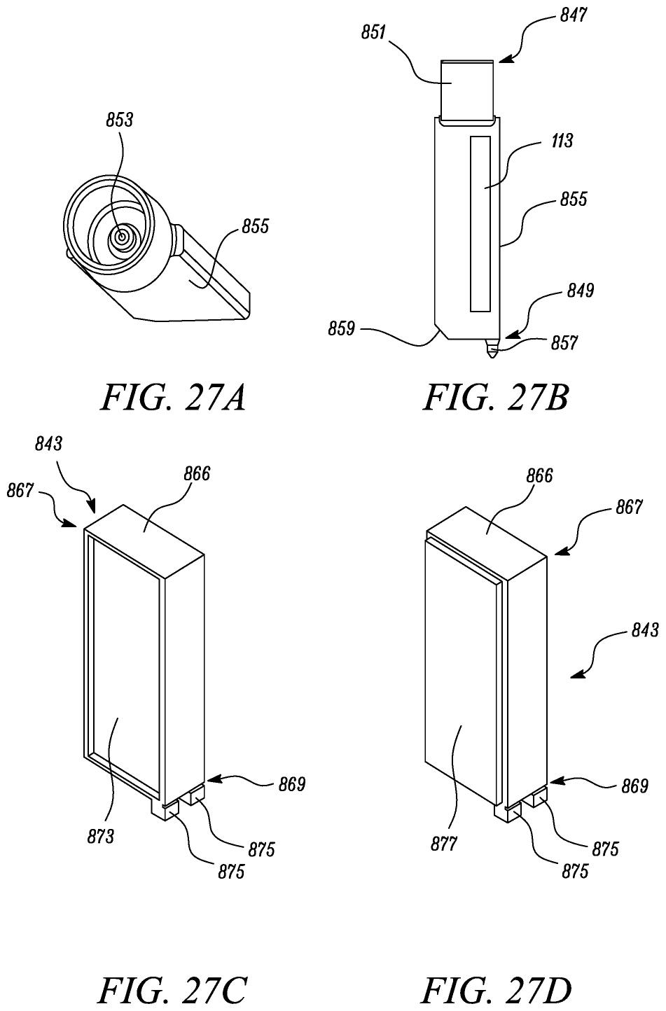

As shown in FIGS. 27A-27D, LCAT chip (841) extends from a top (847) to a bottom (849). An input well (851) is disposed at top (847) and used to receive a whole blood sample therein. Input well (851) may be cylindrical shaped and extending upwardly when LCAT chip (841) is disposed in LCAT rack (845) to form a convenient area for receiving the whole blood sample. In some versions of analyzer (601) and LCAT chip (841), the whole blood is deposited into input well (851) from a nozzle, probe, or other transfer element. In other versions of analyzer (601) and LCAT chip (841), a needle (853) is disposed within input well (851) and utilized to puncture or otherwise penetrate a cap of a blood tube (not shown) to facilitate the transfer of whole blood to LCAT chip (841).

As described above with respect to LCAT chip (800), LCAT chip (841) includes a generally rectangular housing (855) corresponding to a chip slot (856) (FIGS. 26A-26B) defined within LCAT rack (845) for receiving housing (855) of LCAT chip (841) therein. LCAT rack (845) further defines an input well receiving space (858) (FIGS. 26A-26B) for receiving portions of input well (851) therein. A notch (859) is provided and defined by housing (855) to ensure proper insertion of LCAT chip (841) into chip slot (856) and orient input well (851) accordingly within input well receiving space (858). A corresponding alignment feature is disposed (not shown) in the internal area defined by chip slot (856) to prevent LCAT chip (841) from being pressed fully into chip slot (856) when oriented incorrectly.

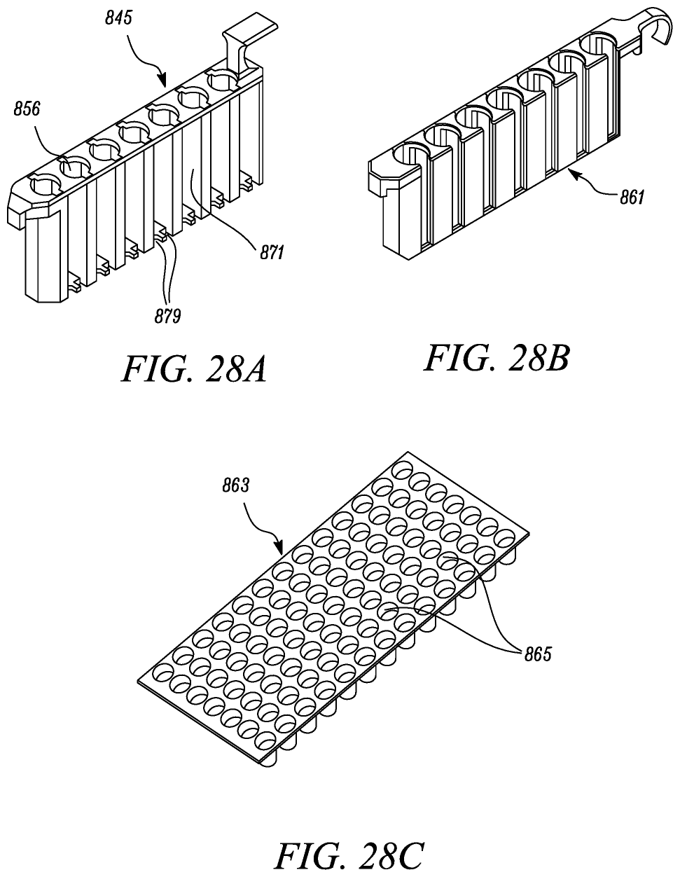

A version of lateral cavity acoustic transducer (LCAT) system (113) is disposed within housing (855), with the input to LCAT system (113) initiating at input well (851). A dispense tip (857) is disposed proximate bottom (849) of LCAT chip (841) and acts as the terminal output of LCAT system (113) disposed within housing (855). Dispense tip (857) is oriented to dispense plasma outwardly away from LCAT chip (841) in a general downward direction when LCAT chip (841) is disposed in LCAT rack (845). While plasma is being produced within the plurality of LCAT chips (841) disposed in a particular LCAT rack (845), a plasma sample rack (861) (FIG. 28B) having a corresponding number of tubes therein (not shown) or a cuvette (863) (FIG. 28C) having a corresponding number of deposit cells (865) may be disposed under each dispense tip (857) in a particular LCAT rack (845) to receive the plasma as it is produced. While dispense tip (857) is shown and described herein, LCAT chip (841) may alternatively include an output well similar to output well (811) described above with respect to LCAT chip (800). Similarly, LCAT chip (800) may include a dispenser tip (857) rather than an output well (811).

As shown in FIGS. 26A-26B and 27A-27D, transducer cartridge (843) includes a housing (866) extending from a top (867) to a bottom (869) and sized to slide into a transducer slot (871) (FIG. 28A) defined by LCAT rack (845). Transducer cartridge (843) further includes a piezo electric material (873) configured to vibrate and create acoustic waves upon activation through electrical power. In some cases, an acoustic conducting material (not shown) may be placed on the piezo electric material to facilitate the transfer of the acoustic energy to LCAT chip (841). An example of this conducting material is TheraSonic Ultrasound Gel. Piezo electric material (873) is operably connected to two leads (875) disposed at bottom (869) of transducer cartridge (843). Leads (875) are configured to receive electric power and pass it to piezo electric material (873) to energize piezo electric material (873). In some version of transducer cartridge (843), a pad (877) is provided adjacent piezo electric material (873) to adjust the acoustic energy emanating from piezo electric material (873). The adjustment may be an enhancement or a dampening of the energy, depending on the preferred acoustic result and materials selected by the user.

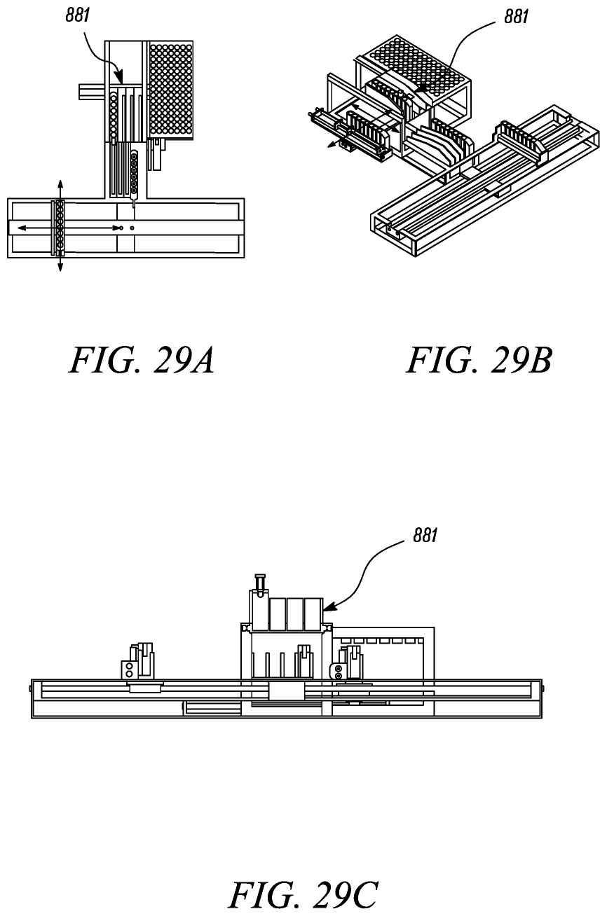

As described above, leads (875) transfer electric power to piezo electric material (873) to produce acoustic energy for agitating LCAT system (113) disposed in housing (855) of LCAT chip (841). As shown in FIGS. 28A, 28B and 28C, LCAT rack (845) defines corresponding lead slots (879) to allow leads (875) to fit therein when transducer cartridge (843) is placed within LCAT rack (845). Corresponding circuitry and electrical transfer elements (not shown) are provided within a transducing module (881) (FIGS. 29A, 29B and 29C) which may be located anywhere within analyzer (601) and used to provide electrical power to each transducer cartridge (843) within a particular LCAT rack (845) undergoing plasma generation.

LCAT chips (841) and transducer cartridges (843) are placed into the associated chip slot (856) and transducer slot (871), respectively, to load LCAT rack (845). Once loaded, LCAT rack (845) is placed within analyzer (601) for use as needed. Input wells (851) of LCAT chips (841) are filled with whole blood and adjacent transducer cartridges (843) are energized to stimulate the production of plasma via LCAT system (113) disposed in housing (855) of LCAT chips (841). Plasma exits LCAT chips (841) via dispenser tips (857) and drips into collection elements disposed below. Thereafter, the collected plasma is transferred as needed throughout analyzer (601) in furtherance of the analyzer workflow.

An exemplary architecture for some version of separation system (607) and for utilizing LCAT rack (845) with one or more LCAT chips (841) and transducer cartridges (843) disposed therein is shown in FIGS. 29A, 29B and 29C. Various transport modules and loading areas are provided for receiving empty LCAT chips (841), transducer cartridges (843), plasma sample racks (861) and/or cuvettes (863) for generating and storing plasma via the above described mechanisms. For example, a user may load LCAT rack (845) with LCAT chips (841) and transducer cartridges (843) as well as a rack filled with whole blood samples into separation system (607). Once initiated, the whole blood will be transferred using various components of movement system (609) to deposit whole blood samples into LCAT chips (841) within LCAT rack (845). LCAT rack (845) is thereafter moved to transducing module (881) where leads of the individual transducer cartridges (843) abut electrical elements provided by transducing module (881). Piezo electric material (873) within each transducer cartridge (843) is thereafter energized to produce acoustic energy, which stimulates and vibrates LCAT system (113) within each adjacent LCAT chip (841). The acoustic energy produces plasma from the whole blood sample, which moves through LCAT system (113) and into dispensing tip (857). Plasma sample rack (861) is positioned below LCAT rack (845) and receives the dripping plasma into plasma sample tubes (not shown). Plasma samples within plasma sample rack (861) are thereafter used as needed within analyzer (601). Thereafter, used LCAT chips (841) may be disposed of and LCAT rack (845) may be reloaded by the user with new LCAT chips (841). Transducer cartridges (843) are generally reusable in some versions of analyzer system (602), though disposable transducer cartridges (843) may be used as desired or needed.

VII. Exemplary Method of Analyzing a Whole Blood Sample

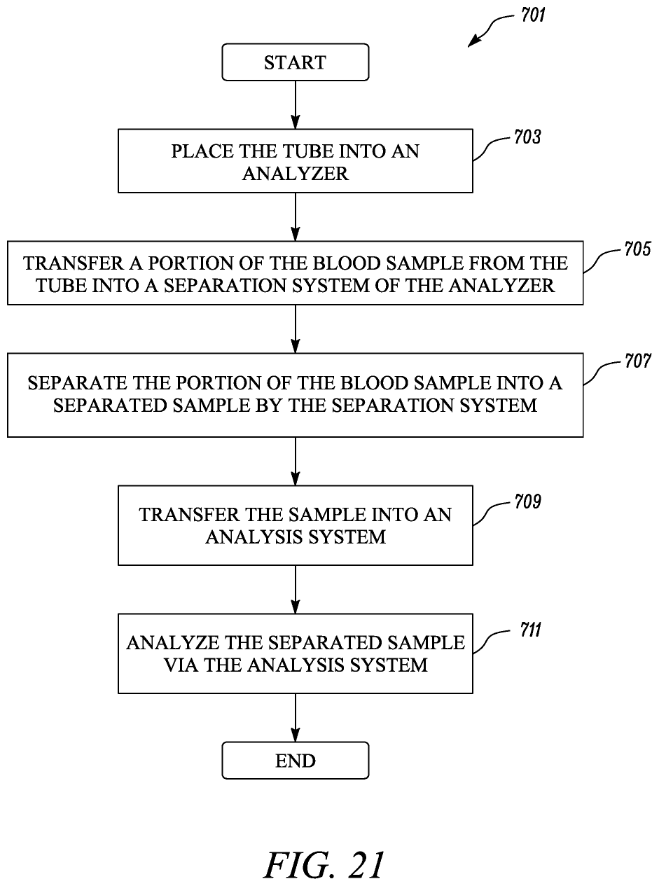

One method of analyzing a whole blood sample (701) using some of the elements described above is depicted generally in FIG. 21. Method of analyzing a blood sample (701) begins with a step (703), whereby a tube similar to tube (603) of FIG. 15 is placed into an analyzer similar to analyzer (601) of FIG. 15. The tube contains a whole blood sample from an individual. The placement of the tube may be performed by a nurse or phlebotomist or any other caregiver or clinician. Once the tube is placed into the analyzer, method of analyzing a blood sample (701) proceeds from step (703) to a step (705).

In step (705), all or some of the whole blood sample from the tube is transferred into a separation system of the analyzer, wherein the separation system is similar to separation system (607) of FIG. 15. The transfer of the whole blood sample from the tube to the separation system may be accomplished by a movement system similar to movement system (609) of FIG. 15. For example, a first transfer element such as a movable pipette may be used to draw all or some of the whole blood sample from the tube and deposit it into the separation system. After the whole blood sample is transferred from the tube to the separation system, method of analyzing a blood sample (101) proceeds from step (705) to a step (707).

In step (707), the whole blood sample deposited in the separation system is separated into a separated sample without the use of centrifugation. The separated sample may comprise plasma or serum. As described with separation system (607), the blood sample may be separated into the separated sample by way of a microfluidic separation system such as an LCAT system or similar non-centrifugation mechanisms. Step (707) may make use of a cassette, similar to cassette (617) of FIGS. 15 and 16, to separate the whole blood sample into the separated sample. The cassette may be removable and replaceable within separation system and include one or more separation channels similar to separation channel (623) described above and depicted in FIG. 16. After the separated sample is separated from the whole blood sample, method of analyzing a blood sample (701) proceeds from step (707) to a step (709).

In step (709), the separated sample generated in step (707) is transferred into an analysis system similar to analysis system (611) as described above. The transfer of the separated sample from the separation system to the analysis system may be accomplished by a movement system similar to movement system (609) of FIG. 15. For example, a second transfer element such as a movable pipette may be used to draw the separated sample from the separation system and deposit it into the analysis system. After the separated sample is deposited in the analysis system, method of analyzing a blood sample (701) proceeds from step (709) to a step (711).

In step (711), the separated sample is analyzed by the analysis system. The analysis system may include a chemistry analyzer, an immunoassay analyzer, a molecular analyzer, a mass spectrometry analyzer, or a combination thereof. After the separated sample is analyzed by the analysis system, method of analyzing a blood sample (701) proceeds to end.

VIII. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A collection system comprising: (a) a tube; and (b) a holder, wherein the holder is configured to receive the tube therein.

### Example 2

The collection system of Example 1 or any of the subsequent Examples, wherein the tube comprises a plasma separation system.

### Example 3

The collection system or methods of any of the previous or subsequent Examples, wherein the plasma separation system comprises a microfluidic separation system.

### Example 4

The collection system or methods of any of the previous or subsequent Examples, wherein the plasma separation system comprises a lateral cavity acoustic transducer.

### Example 5

The collection system or methods of any of the previous or subsequent Examples, wherein the lateral cavity acoustic transducer system comprises (a) a main channel; (b) a lateral cavity extending from the main channel along a non-orthogonal axis; and (c) a transducer configured to oscillate fluid within the main channel and the lateral cavity.

### Example 6

The collection system or methods of any of the previous or subsequent Examples, wherein the tube defines a collection chamber.

### Example 7

The collection system or methods of any of the previous or subsequent Examples, wherein the collection chamber defines an analysis pocket therein.

### Example 8

The collection system or methods of any of the previous or subsequent Examples, wherein the holder comprises an imaging system.

### Example 9

The collection system or methods of any of the previous or subsequent Examples, wherein an optics element of the imaging system is directed at the analysis pocket when the tube is disposed in the holder.

### Example 10

The collection system or methods of any of the previous or subsequent Examples, wherein a portion of the tube adjacent the analysis pocket is transparent.

### Example 11

The collection system or methods of any of the previous or subsequent Examples, wherein the holder comprises a reagent reservoir, wherein the holder is configured to transfer a reagent from the reagent reservoir to the collection chamber.

### Example 12

The collection system or methods of any of the previous or subsequent Examples, wherein the tube comprises a label area on an outer surface.

### Example 13

The collection system or methods of any of the previous or subsequent Examples, wherein the holder comprises a labeling system, wherein the labeling system is configured to apply a label to the label area when the tube is disposed in the holder.

### Example 14

The collection system or methods of any of the previous or subsequent Examples, wherein the label area comprises a flat rectangular-shaped surface.

### Example 15

The collection system or methods of any of the previous or subsequent Examples, wherein the tube comprises a tube alignment element, wherein the holder includes a holder alignment element, wherein the tube alignment element is configured to cooperate with the holder alignment element when the tube is disposed in the holder.

### Example 16

A collection system comprising: (a) a tube comprising: (i) a collection chamber, (ii) a plasma separation system, wherein the plasma separation system is disposed in the collection chamber, and (iii) an analysis pocket extending from the collection chamber, wherein the analysis pocket is configured to receive therein a unit of plasma provided from the plasma separation system; and (b) a holder configured to draw power from a power supply, the holder comprising: (i) a display screen, wherein the display screen is powered by the power supply; (ii) a tube receptacle, wherein the tube receptacle is configured to receive the tube therein, (iii) an analysis system, wherein the analysis system is configured to collect a set of data from the unit of plasma disposed in the analysis pocket, wherein the analysis system is configured to actuate the display screen to display the set of data, (iv) a labeling system, wherein the labeling system is configured to apply a label to the tube, and (v) a transducer configured to oscillate fluid within a main channel and a lateral cavity of the plasma separation system.

### Example 17

The collection system or methods of any of the previous or subsequent Examples, wherein the plasma separation system comprises a microfluidic separation system.

### Example 18

The collection system or methods of any of the previous or subsequent Examples, wherein the microfluidic separation system comprises a lateral cavity acoustic transducer system.

### Example 19

The collection system or methods of any of the previous or subsequent Examples, the holder comprising a reagent reservoir, wherein the holder is configured to transfer a reagent from the reagent reservoir to the collection chamber.

### Example 20

The collection system or methods of any of the previous or subsequent Examples, wherein the tube holder comprises a lateral cavity acoustic transducer system.

### Example 21

A method comprising: (a) placing a blood sample into a tube; (b) receiving the tube in a holder; (c) oscillating the blood sample in the tube with a transducer; (d) in response to oscillating the blood sample in the tube with the transducer, creating a plasma sample from the blood sample within the tube; and (e) conducting spectroscopic or imaging analysis on the plasma sample with an analysis system of the holder to derive a sample quality.

### Example 22

The collection system or methods of any of the previous or subsequent Examples, further comprising indicating the sample quality to a user on a display screen of the holder.

### Example 23

The collection system or methods of any of the previous or subsequent Examples, further comprising indicating the sample quality to the user on an indication system of the holder.

### Example 24

The collection system or methods of any of the previous or subsequent Examples, further comprising providing the sample quality to a central analysis platform.

### Example 25

The collection system or methods of any of the previous or subsequent Examples, further comprising labeling the tube while the tube is disposed within the holder.

### Example 26

The collection system or methods of any of the previous or subsequent Examples, further comprising programming an RFID chip embedded within the tube while the tube is disposed within the holder.

### Example 27

The collection system or methods of any of the previous or subsequent Examples, further comprising creating the plasma sample via a plasma separation system disposed in the tube.

### Example 28

The collection system or methods of any of the previous or subsequent Examples, wherein the plasma separation system comprises a microfluidic separation system.

### Example 29

The collection system or methods of any of the previous or subsequent Examples, wherein the microfluidic separation system comprises a lateral cavity acoustic transducer system.

### Example 30

The collection system or methods of any of the previous or subsequent Examples, wherein the transducer is disposed in the holder.

### Example 31

The collection system or methods of any of the previous or subsequent Examples, wherein the tube comprises an electronic label.

### Example 32

The collection system or methods of any of the previous or subsequent Examples, wherein the holder comprises a labeling system, wherein the labeling system is configured to program the electronic label when the tube is disposed in the holder.

### Example 33

The collection system or methods of any of the previous Examples, further comprising indicating the sample quality to a user

### Example 34

A device comprising: (a) a main body; (b) a needle attachment feature, wherein the needle attachment feature is configured to couple a needle element to the main body, wherein the needle attachment feature is configured to transmit a blood sample from the needle element into the main body; (c) a first chamber; (d) a second chamber; and (e) a separation element, wherein the separation element is configured to separate the blood sample into a first portion and a second portion, wherein the separation element is configured to transmit the first portion into the first chamber, wherein the separation element is configured to transmit the second portion into the second chamber.

### Example 35

The device, system, and/or method of the previous or subsequent Examples, wherein the separation element is disposed in the main body.

### Example 36

The device, system, and/or method of any of the previous or subsequent Examples, wherein one or both of the first chamber and the second chamber are removably secured to the main body.

### Example 37

The device, system, and/or method of any of the previous or subsequent Examples, wherein one or both of the first chamber and the second chamber comprise a test tube.

### Example 38

The device, system, and/or method of any of the previous or subsequent Examples, wherein one of the first chamber and the second chamber includes an interior surface, wherein the interior surface is coated with silica.

### Example 39

The device, system, and/or method of any of the previous or subsequent Examples, wherein one of the first chamber and the second chamber includes an interior surface, wherein the interior surface is coated with an anticoagulant.

### Example 40

The device, system, and/or method of any of the previous or subsequent Examples, wherein the separation element comprises a plasma separation system.

### Example 41

The device, system, and/or method of any of the previous or subsequent Examples, wherein the plasma separation system comprises a microfluidic separation system.

### Example 42

The device, system, and/or method of any of the previous or subsequent Examples, wherein the microfluidic separation system comprises a lateral cavity acoustic transducer system.

### Example 43

The device, system, and/or method of any of the previous or subsequent Examples, wherein the lateral cavity acoustic transducer system comprises: (a) a main channel; (b) a lateral cavity extending from the main channel along a non-orthogonal axis; and (c) a transducer configured to oscillate fluid within the main channel and the lateral cavity.

### Example 44

The device, system, and/or method of any of the previous or subsequent Examples, wherein the separation element comprises one or more of a micro-channel, a filter, an anticoagulant, and a resin.

### Example 45

The device, system, and/or method of any of the previous or subsequent Examples, wherein the needle element comprises a blood draw needle.

### Example 46

The device, system, and/or method of any of the previous or subsequent Examples, wherein the needle element comprises a finger stick attachment.

### Example 47

The device, system, and/or method of any of the previous or subsequent Examples, comprising an identification element associated with the main body.

### Example 48

The device, system, and/or method of any of the previous or subsequent Examples, wherein the separation element is configured to separate the blood sample into the first portion based at least in part on the identification element.

### Example 49

The device, system, and/or method of any of the previous or subsequent Examples, wherein the first chamber is removably secured to the main body.

### Example 50

A method comprising: (a) drawing a blood sample into a device; (b) separating the blood sample into a sample of whole blood, a sample of plasma, and a sample of serum; (c) transmitting the sample of whole blood into a first chamber connected to the device; (d) transmitting the sample of plasma into a second chamber connected to the device; and (e) transmitting the sample of serum into a third chamber connected to the device.

### Example 51

The device, system, and/or method of any of the previous or subsequent Examples, further comprising disconnecting one or more of the first chamber, the second chamber, and the third chamber from the device.

### Example 52

The device, system, and/or method of any of the previous or subsequent Examples, further comprising: (a) aspirating the blood sample through a needle element into a separation element of the device; and (b) transmitting the sample of blood through the separation element to separate the blood sample into the sample of whole blood, the sample of plasma, and the sample of serum.

### Example 53

The device, system, and/or method of any of the previous or subsequent Examples, wherein the needle element comprises a blood draw needle.

### Example 54

The device, system, and/or method of any of the previous or subsequent Examples, wherein the needle element comprises a finger stick attachment.

### Example 55

The device, system, and/or method of any of the previous or subsequent Examples, further comprising coating an interior surface of the first chamber with an anticoagulant.

### Example 56

The device, system, and/or method of any of the previous or subsequent Examples, further comprising coating an interior surface of the third chamber with silica.

### Example 57

The device, system, and/or method of any of the previous or subsequent Examples, further comprising: (a) prior to drawing the blood sample into the device, connecting a syringe to the device; (b) in response to connecting the syringe to the device, creating a vacuum in the device.

### Example 58

The device, system, and/or method of any of the previous or subsequent Examples, further comprising after transmitting the sample of whole blood into the first chamber connected to the device, the sample of plasma into the second chamber connected to the device, and the sample of serum into the third chamber connected to the device, disconnecting the syringe from the device.

### Example 59

The device, system, and/or method of any of the previous or subsequent Examples, further comprising after disconnecting the syringe from the device, loading the device into a sample rack.

### Example 60

The device, system, and/or method of any of the previous or subsequent Examples, further comprising inserting a first portion of the device into a slot defined by the sample rack to load the device into the sample rack, wherein the syringe is connected to the first portion of the device.

### Example 61

A system comprising: (a) a device comprising: (i) a main body, (ii) a needle attachment feature, wherein the needle attachment feature is configured to selectively couple a needle element to the main body, wherein the needle attachment feature is configured to transmit a blood sample from the needle element into the main body, and (iii) a separation element disposed inside the main body, wherein the separation element is configured to receive the blood sample and separate the blood sample into a sample of whole blood, a sample of plasma, and a sample of serum; (b) a first sample tube, wherein the first sample tube is removably connected to the device, wherein the first sample tube is configured to receive the sample of whole blood from the device; (c) a second sample tube, wherein the second sample tube is removably connected to the device, wherein the second sample tube is configured to receive the sample of plasma from the device; and (d) a third sample tube, wherein the third sample tube is removably connected to the device, wherein the third sample tube is configured to receive the sample of serum from the device.

### Example 62

The device, system, and/or method of any of the previous or subsequent Examples, wherein an interior surface of the first sample tube includes a layer of anticoagulant.

### Example 63

The device, system, and/or method of any of the previous or subsequent Examples, wherein an interior surface of the third sample tube includes a layer of silica.

### Example 64

The device, system, and/or method of any of the previous or subsequent Examples, wherein the separation element comprises one or more of a micro-channel, a filter, an anticoagulant, and a resin.

### Example 65

The device, system, and/or method of any of the previous or subsequent Examples, wherein the separation element comprises a plasma separation system.

### Example 66

The device, system, and/or method of any of the previous or subsequent Examples, wherein the plasma separation system comprises a microfluidic separation system.

### Example 67

The device, system, and/or method of any of the previous or subsequent Examples, wherein the microfluidic separation system comprises a lateral cavity acoustic transducer system.

### Example 68

The device, system, and/or method of any of the previous or subsequent Examples, wherein the lateral cavity acoustic transducer system comprises: (a) a main channel; (b) a lateral cavity extending from the main channel along a non-orthogonal axis; and (c) a transducer configured to oscillate fluid within the main channel and the lateral cavity.

### Example 69

The device, system, and/or method of any of the previous or subsequent Examples, wherein the needle element comprises a blood draw needle.

### Example 70

The device, system, and/or method of any of the previous or subsequent Examples, wherein the needle element comprises a finger stick attachment.

### Example 71

An analyzer configured to receive a tube containing a blood sample, the analyzer comprising: (a) a separation system; (b) a transfer element, wherein the transfer element is configured to obtain at least a portion of the blood sample from the tube, wherein the transfer element is configured to deposit the portion of the blood sample into the separation system; (c) a movement system, wherein the movement system is configured to move the transfer element between the tube and the separation system; and wherein the separation system is configured to separate the portion of the blood sample into a separated sample, wherein the separated sample comprises one of a plasma sample and a serum sample.

### Example 72

The analyzer, analyzer system, cassette, and/or method of the previous or any of the subsequent Examples, wherein the transfer element is a first transfer element and further comprising: (a) an analysis system; (b) a second transfer element, wherein the second transfer element is configured to obtain at least a portion of the separated sample from the separation system, wherein the second transfer element is configured to deposit the portion of the separated sample into the analysis system; and wherein the movement system is configured to move the second transfer element between the separation system and the analysis system.

### Example 73

The analyzer, analyzer system, cassette, and/or method of any of the previous or subsequent Examples, wherein the analysis system comprises one of a spectroscopic analyzer, a thermocycler element for PCR, an isotheral amplification element, an immune assay, and an Elisa system.

### Example 74

The analyzer, analyzer system, cassette, and/or method of any of the previous or subsequent Examples, wherein one of the first transfer element and the second transfer element comprises a pipette.

### Example 75

The analyzer, analyzer system, cassette, and/or method of any of the previous or subsequent Examples, wherein the separation system a microfluidic separation system.

### Example 76

The analyzer, analyzer system, cassette, and/or method of any of the previous or subsequent Examples, wherein the microfluidic separation system comprises a lateral cavity acoustic transducer system.

### Example 77

The analyzer, analyzer system, cassette, and/or method of any of the previous or subsequent Examples, wherein the lateral cavity acoustic transducer system comprises: (a) a main channel; (b) a lateral cavity extending from the main channel along a non-orthogonal axis; and (c) a transducer configured to oscillate fluid within the main channel and the lateral cavity.

### Example 78

The analyzer, analyzer system, cassette, and/or method of any of the previous or subsequent Examples, wherein the separation system defines a cassette receptacle, wherein the cassette receptacle is configured to receive a cassette therein.

### Example 79

An analyzer system comprising: (a) a cassette; and (b) an analyzer configured to receive a tube containing a blood sample and the cassette, the analyzer comprising: (i) a separation system; (ii) a transfer element, wherein the transfer element is configured to obtain at least portion of the blood sample from the tube, wherein the transfer element is configured to deposit the portion of the blood sample into the separation system; (iii) a movement system, wherein the movement system is configured to move the transfer element between the tube and the separation system; (iv) a cassette receptacle defined by the separation system, wherein the cassette receptacle is configured to removably receive the cassette therein; and wherein the separation system is configured to separate the portion of the blood sample into a separated sample via the cassette, wherein the separated sample comprises one of a plasma sample and a serum sample.

### Example 80

The analyzer, analyzer system, cassette, and/or method of any of the previous or subsequent Examples, wherein the cassette comprises a plurality of separation channels, wherein the separation system is configured to separate the portion of the blood sample into the separated sample via one of the plurality of separation channels.

### Example 81

The analyzer, analyzer system, cassette, and/or method of any of the previous or subsequent Examples, wherein each separation channel in the plurality of separation channels comprises: (a) a first portion, wherein the first portion is configured to receive the portion of the blood sample therein; and (b) a second portion, wherein the second portion is configured to receive the portion of the blood sample from the first portion and separate the portion of the blood sample into the separated sample.

### Example 82

The analyzer, analyzer system, cassette, and/or method of any of the previous or subsequent Examples, wherein the second portion comprises a microfluidic separation system.

### Example 83

The analyzer, analyzer system, cassette, and/or method of any of the previous or subsequent Examples, wherein the microfluidic separation system comprises a lateral cavity acoustic transducer system.

### Example 84

The analyzer, analyzer system, cassette, and/or method of any of the previous or subsequent Examples, wherein the lateral cavity acoustic transducer system comprises: (a) a main channel; (b) a lateral cavity extending from the main channel along a non-orthogonal axis; and (c) a transducer configured to oscillate fluid within the main channel and the lateral cavity.

### Example 85

A method of analyzing a blood sample in a tube, the method comprising: (a) placing the tube into an analyzer; (b) transferring at least a portion of the blood sample from the tube into a separation system of the analyzer, wherein the transferring is performed automatically within the analyzer; (c) separating the portion of the blood sample into a separated sample by the separation system, wherein the separated sample is one of a plasma sample and a serum sample; (d) transferring the separated sample into an analysis system, wherein the transferring is performed automatically within the analyzer; and (e) analyzing the separated sample via the analysis system.

### Example 86

The analyzer, analyzer system, cassette, and/or method of any of the previous or subsequent Examples, wherein the separation system comprises a removable cassette, and further comprising transferring the portion of the blood sample from the tube into the cassette.

### Example 87

The analyzer, analyzer system, cassette, and/or method of any of the previous or subsequent Examples, wherein the cassette comprises at least one sealed well, and further comprising: (a) puncturing one of the at least one sealed wells to form a punctured well; and (b) transferring the portion of the blood sample from the tube into the punctured well.

### Example 88

The analyzer, analyzer system, cassette, and/or method of any of the previous or subsequent Examples, wherein the cassette comprises a separation portion and at least one open well, and further comprising moving the portion of the blood sample from the punctured well into the separation portion and toward one of the at least one open wells to separate the portion of the blood sample into the separated sample.

### Example 89

The analyzer, analyzer system, cassette, and/or method of any of the previous or subsequent Examples, wherein the separation system comprises a microfluidic separation system.

### Example 90

The analyzer, analyzer system, cassette, and/or method of any of the previous or subsequent Examples, wherein the microfluidic separation system comprises a lateral cavity acoustic transducer system.

### Example 91

The analyzer, analyzer system, cassette, and/or method of any of the previous or subsequent Examples, wherein the lateral cavity acoustic transducer system comprises: (a) a main channel; (b) a lateral cavity extending from the main channel along a non-orthogonal axis; and (c) a transducer configured to oscillate fluid within the main channel and the lateral cavity.

### Example 92

The analyzer, analyzer system, cassette, and/or method of any of the previous or subsequent Examples, further comprising: (a) transferring the portion of the blood sample from the tube into a first portion of the cassette; (b) separating the portion of the blood sample into the separated sample in a second portion of the cassette; and (c) transferring the separated sample from a third portion of the cassette into the analysis system.

### Example 93

The analyzer, analyzer system, cassette, and/or method of any of the previous or subsequent Examples, further comprising puncturing the first portion prior to transferring the portion of the blood sample from the tube into the first portion of the cassette.

### Example 94

The analyzer, analyzer system, cassette, and/or method of any of the previous or subsequent Examples, wherein the second portion comprises a microfluidic separation system.

### Example 95

The analyzer, analyzer system, cassette, and/or method of any of the previous or subsequent Examples, wherein the cassette comprises a plurality of separation channels, wherein each separation channel in the plurality of separation channels comprises a first portion and a second portion, and further comprising: (a) selecting a separation channel from the plurality of separation channels; (b) transferring the portion of the blood sample from the tube into the first portion of the selected separation channel; and (c) separating the portion of the blood sample into the separated sample in the second portion of the selected separation channel.

### Example 96

The analyzer, analyzer system, cassette, and/or method of any of the previous or subsequent Examples, wherein each separation channel in the plurality of separation channels comprises a third portion, and further comprising transferring the separated sample from the third portion of the selected separation channel into the analysis system.

### Example 97

The analyzer, analyzer system, cassette, and/or method of any of the previous or subsequent Examples, further comprising puncturing the first portion of the selected separation channel prior to transferring the portion of the blood sample from the tube into the first portion of the selected separation channel.

### Example 98

The analyzer, analyzer system, cassette, and/or method of any of the previous or subsequent Examples, wherein the second portion comprises a microfluidic separation system.

### Example 99

The analyzer, analyzer system, cassette, and/or method of any of the previous or subsequent Examples, wherein the second portion comprises a lateral cavity acoustic transducer system.

### Example 100

The analyzer, analyzer system, cassette, and/or method of any of the previous or subsequent Examples, further comprising manually removing the cassette from the analyzer.

### Example 101

A cassette configured to be selectively disposed in an analyzer, the cassette comprising:

(a) a main body; (b) at least one separation channel disposed within the main body, wherein each separation channel is configured to separate at least a portion of a blood sample into a separated sample.

### Example 102

The analyzer, analyzer system, cassette, and/or method of any of the previous or subsequent Examples, wherein each separation channel comprises: (a) a first portion, wherein the first portion is configured to receive the portion of the blood sample therein; and (b) a second portion, wherein the second portion is configured to receive the portion of the blood sample from the first portion and separate the portion of the blood sample into the separated sample.

### Example 103

The analyzer, analyzer system, cassette, and/or method of any of the previous or subsequent Examples, wherein the second portion comprises a microfluidic separation system.

### Example 104

The analyzer, analyzer system, cassette, and/or method of any of the previous or subsequent Examples, wherein the microfluidic separation system comprises a lateral cavity acoustic transducer system.

### Example 105

The analyzer, analyzer system, cassette, and/or method of any of the previous or subsequent Examples, wherein the lateral cavity acoustic transducer system comprises: (a) a main channel; (b) a lateral cavity extending from the main channel along a non-orthogonal axis; and (c) a transducer configured to oscillate fluid within the main channel and the lateral cavity.

### Example 106

The analyzer, analyzer system, cassette, and/or method of any of the previous or subsequent Examples, wherein the first portion is a sealed well configured to be punctured prior to receiving the portion of the blood sample therein.

### Example 107

The analyzer, analyzer system, cassette, and/or method of any of the previous or subsequent Examples, wherein each separation channel comprises a third portion configured to receive the separated sample from the second portion.

### Example 108

The analyzer, analyzer system, cassette, and/or method of any of the previous or subsequent Examples, wherein the third portion is an open well.

### Example 109

The analyzer, analyzer system, cassette, and/or method of any of the previous or subsequent Examples, wherein the separated sample is at least 10 microliters.

### Example 110

The analyzer, analyzer system, cassette, and/or method of any of the previous or subsequent Examples, further comprising separating the portion of the blood sample into multiple separated samples by the separation system.

### Example 111

The analyzer, analyzer system, cassette, and/or method of any of the previous or subsequent Examples, further comprising transferring a first separated sample in the multiple separated samples into a chemistry analyzer, wherein the transferring of the first separated sample is performed automatically within the chemistry analyzer.

### Example 112

The analyzer, analyzer system, cassette, and/or method of any of the previous or subsequent Examples, further comprising transferring a second separated sample in the multiple separated samples into an immunoassay analyzer, wherein the transferring of the second separated sample is performed automatically within the immunoassay analyzer.

### Example 113

The analyzer, analyzer system, cassette, and/or method of any of the previous or subsequent Examples, further comprising transferring a third separated sample in the multiple separated samples into a molecular analyzer, wherein the transferring of the third separated sample is performed automatically within the molecular analyzer.

### Example 114

The analyzer, analyzer system, cassette, and/or method of any of the previous or subsequent Examples, further comprising transferring a fourth separated sample in the multiple separated samples into a mass spectrometry analyzer, wherein the transferring of the fourth separated sample is performed automatically within the mass spectrometry analyzer.

### Example 115

The analyzer, analyzer system, cassette, and/or method of any of the previous or subsequent Examples, further comprising transferring the second portion of the blood sample from the tube into a storage container.

### Example 116

The analyzer, analyzer system, cassette, and/or method of any of the previous or subsequent Examples, further comprising transferring at least portion of the separated sample into a storage container.

### Example 117

The analyzer, analyzer system, cassette, and/or method of any of the previous or subsequent Examples, wherein the microfluidic separation system comprises a lateral cavity acoustic transducer system.

### Example 118

The analyzer, analyzer system, cassette, and/or method of any of the previous or subsequent Examples, wherein the lateral cavity acoustic transducer system comprises (a) a main channel, (b) a lateral cavity extending from the main channel along an axis, and (c) a transducer configured to oscillate fluid within the main channel and the lateral cavity.

### Example 119

The analyzer, analyzer system, cassette, and/or method of any of the previous or subsequent Examples, wherein the microfluidic separation system comprises a lateral cavity acoustic transducer system.

### Example 120

The analyzer, analyzer system, cassette, and/or method of any of the previous or subsequent Examples, wherein the second portion comprises a lateral cavity acoustic transducer system.

### Example 121

The analyzer, analyzer system, cassette, and/or method of any of the previous or subsequent Examples, wherein the lateral cavity acoustic transducer system comprises (a) a main channel, (b) a lateral cavity extending from the main channel along an axis, and (c) a transducer configured to oscillate fluid within the main channel and lateral cavity.

### Example 122

An analyzer system comprising (a) a transducer, (b) an LCAT chip configured to receive a whole blood sample and produce a plasma sample in response to stimulation by the transducer, (c) an LCAT rack, wherein the LCAT rack is configured to removably receive the LCAT chip therein, and wherein the transducer is configured to stimulate the LCAT chip to produce plasma while the LCAT chip is in the LCAT rack.

### Example 123

The analyzer, analyzer system, cassette, and/or method of any of the previous or subsequent Examples, the LCAT chip comprising an input well configured to receive the whole blood sample therein.

### Example 124

The analyzer, analyzer system, cassette, and/or method of any of the previous or subsequent Examples, the LCAT rack defining an input well receiving space, wherein the input well is disposed within the input well receiving space when the LCAT chip is disposed within the LCAT rack.

### Example 125

The analyzer, analyzer system, cassette, and/or method of any of the previous or subsequent Examples, the LCAT chip comprising an output well configured to collect the plasma generated therein.

### Example 126

The analyzer, analyzer system, cassette, and/or method of any of the previous or subsequent Examples, the LCAT rack defining an output well receiving space, wherein the output well is disposed within the output well receiving space when the LCAT chip is disposed within the LCAT rack.

### Example 127

The analyzer, analyzer system, cassette, and/or method of any of the previous or subsequent Examples, the LCAT chip comprising a dispense tip configured to expel the plasma generated therein.

### Example 128

The analyzer, analyzer system, cassette, and/or method of any of the previous or subsequent Examples, the LCAT chip comprising a housing defining a notch, the LCAT rack comprising an alignment feature, wherein the notch and the alignment feature align when the LCAT chip is fully inserted into the LCAT rack.

### Example 129

The analyzer, analyzer system, cassette, and/or method of any of the previous or subsequent Examples, the LCAT rack defining a chip slot therein, wherein the LCAT chip is disposed in the chip slot when the LCAT chip is disposed within the LCAT rack.

### Example 130

The analyzer, analyzer system, cassette, and/or method of any of the previous or subsequent Examples, wherein the transducer is disposed in a plasma separation module, wherein the plasma separation module defines a transducer slot sized to receive the LCAT rack.

### Example 131

The analyzer, analyzer system, cassette, and/or method of any of the previous or subsequent Examples, wherein the transducer is disposed in the LCAT rack.

### Example 132

The analyzer, analyzer system, cassette, and/or method of any of the previous or subsequent Examples, wherein the transducer is disposed within a transducer cartridge, wherein the LCAT rack is configured to removably receive the transducer cartridge therein.

### Example 133

The analyzer, analyzer system, cassette, and/or method of any of the previous or subsequent Examples, wherein the transducer cartridge comprises a lead, wherein the lead is operably coupled with the transducer.

### Example 134

The analyzer, analyzer system, cassette, and/or method of any of the previous or subsequent Examples, wherein the LCAT rack defines a lead slot, wherein the lead is disposed within the lead slot when the transducer cartridge is disposed within the LCAT rack.

### Example 135

The analyzer, analyzer system, cassette, and/or method of any of the previous or subsequent Examples, further comprising a transducing module, wherein the transducing module is configured to provide electrical power to the lead of the transducer cartridge.

### Example 136

The analyzer, analyzer system, cassette, and/or method of any of the previous or subsequent Examples, the transducer further comprising piezo electric material.

Other contemplated embodiments are described in the following paragraph.

1. A collection system (1) comprising:

(a) a tube (102); and (b) a holder (104), wherein the holder (104) is configured to receive the tube (102) therein.

2. The collection system (1) of claim 1, further comprising a plasma separation system (114).

3. The collection system (1) of claim 2, wherein the plasma separation system (114) comprises a microfluidic separation system.

4. The collection system (1) of claim 3, wherein the microfluidic separation system comprises a lateral cavity acoustic transducer system (113).

5. The collection system (1) of claim 4, wherein the lateral cavity acoustic transducer system (113) comprises: (a) a main channel (127); (b) a lateral cavity (121) extending from the main channel (127) along a non-orthogonal axis; and=(c) a transducer (123) configured to oscillate fluid within the main channel (127) and the lateral cavity (121).

6. The collection system (1) of any one of claims 1 to 5, wherein the tube (102) defines a collection chamber (112).

7. The collection system (1) of claim 6, wherein the collection chamber (112) defines an analysis pocket (116) therein.

8. The collection system (1) of any one of claims 1 to 7, wherein the holder (104) comprises an imaging system.

9. The collection system (1) of claim 8, wherein an optics element of the imaging system is directed at the analysis pocket (116) when the tube (102) is disposed in the holder (104).

10. The collection system (1) of any one of claims 7 to 9, wherein a portion of the tube (102) adjacent the analysis pocket (116) is transparent.

11. The collection system (1) of any one of claims 6 to 10, wherein the holder (104) comprises a reagent reservoir (138), wherein the holder (104) is configured to transfer a reagent from the reagent reservoir (138) to the collection chamber (112).

12. The collection system (1) of any one of claims 1 to 10, wherein the tube (102) comprises a label area (120) on an outer surface.

13. The collection system (1) of claim 12, wherein the holder (104) comprises a labeling system (142), wherein the labeling system (142) is configured to apply a label (122) to the label area (120) when the tube (102) is disposed in the holder (104).

14. The collection system (1) of claim 12 or 13, wherein the label area (120) comprises a flat rectangular-shaped surface.

15. The collection system (1) of any one of the claims 1 to 13, wherein the tube (102) comprises a tube alignment element (124), wherein the holder (104) includes a holder alignment element (130), wherein the tube alignment element (124) is configured to cooperate with the holder alignment element (130) when the tube (102) is disposed in the holder (104).

16. A collection system (1) comprising: (a) a tube (102) comprising: (i) a collection chamber (112), (ii) a plasma separation system (114), wherein the plasma separation system (114) is disposed in the collection chamber (112), and (iii) an analysis pocket (116) extending from the collection chamber (112), wherein the analysis pocket (116) is configured to receive therein a unit of plasma provided from the plasma separation system (114); and (b) a holder (104) configured to draw power from a power supply (148), the holder (104) comprising: (i) a display (134), wherein the display (134) is powered by the power supply (148), (ii) a tube receptacle (126), wherein the tube receptacle (126) is configured to receive the tube (102) therein, (iii) an analysis system (132), wherein the analysis system (132) is configured to collect a set of data from the unit of plasma disposed in the analysis pocket (116), wherein the analysis system (132) is configured to actuate the display (134) to display the set of data, (iv) a labeling system (142), wherein the labeling system (142) is configured to label the tube (102), and (v) a transducer (123) configured to oscillate fluid within a main channel (127) and a lateral cavity (121) of the plasma separation system (114).

17. The collection system (1) of claim 16, wherein the plasma separation system (114) comprises a microfluidic separation system.

18. The collection system (1) of claim 17, wherein the microfluidic separation system comprises a lateral cavity acoustic transducer system (113).

19. The collection system (1) of any one of claims 16 to 18, the holder (104) comprising a reagent reservoir (138), wherein the holder (104) is configured to transfer a reagent from the reagent reservoir (138) to the collection chamber (112).

20. A method (300) comprising: (a) placing (302) a blood sample into a tube (102);

(b) receiving (306) the tube (102) in a holder (104); (c) oscillating the blood sample in the tube (102) with a transducer (123); (d) in response to oscillating the blood sample in the tube (102) with the transducer (123), creating (304) a plasma sample from the blood sample within the tube (102); and (e) conducting (308) spectroscopic or imaging analysis on the plasma sample with an analysis system (132) of the holder (104) to derive a sample quality.

21. The method (300) of claim 20, further comprising indicating (310) the sample quality to a user on a display screen (134) of the holder (104).

22. The method (300) of claim 20 further comprising indicating the sample quality to a user on an indication system of the holder (104).

23. The method (300) of any one of claims 20 to 22, further comprising providing the sample quality to a central analysis platform.

24. The method (300) of any one of claims 20 to 23, further comprising labeling (316) the tube (102) while the tube (102) is disposed within the holder (104).

25. The method (300) of any one of claims 20 to 24, further comprising programming an RFID chip embedded within the tube (102) while the tube (102) is disposed within the holder (104).

26. The method (300) of any one of claims 20 to 25, further comprising creating the plasma sample via a plasma separation system (114) disposed in the tube (102).

27. The method (300) of claim 26, wherein the plasma separation system (114) comprises a microfluidic separation system.

28. The method (300) of claim 27, wherein the microfluidic separation system comprises a lateral cavity acoustic transducer system (113).

29. The method (300) of any one of claims 20 to 28, wherein the transducer (123) is disposed in the holder (104).

30. The method (300) of any one of claims 20 to 29, wherein the tube (102) comprises an electronic label.

31. The method (300) of claim 30, wherein the holder (104) comprises a labeling system (142), wherein the labeling system (142) is configured to program the electronic label when the tube (102) is disposed in the holder (104).

32. The method (300) of claim 20, further comprising indicating (310) the sample quality to a user.

33. A device (401) comprising: (a) a main body (403); (b) a needle attachment feature (409), wherein the needle attachment feature (409) is configured to couple a needle element (411) to the main body (403), wherein the needle attachment feature (409) is configured to transmit a blood sample from the needle element (411) into the main body (403); (c) a first chamber (413); (d) a second chamber (415); and (e) a separation element (427), wherein the separation element (427) is configured to separate the blood sample into a first portion (429) and a second portion (431), wherein the separation element (427) is configured to transmit the first portion (429) into the first chamber (413), wherein the separation element (427) is configured to transmit the second portion (431) into the second chamber (415).

34. The device (401) of claim 33, wherein the separation element (427) is disposed in the main body (403).

35. The device (401) of claim 33 or 34, wherein one or both of the first chamber (413) and the second chamber (415) are removably secured to the main body (403).

36. The device (401) of any one of claims 33 to 35, wherein one or both of the first chamber (413) and the second chamber (415) comprise a test tube.

37. The device (401) of any one of claims 33 to 36, wherein one of the first chamber (413) and the second chamber (415) includes an interior surface (419, 423), wherein the interior surface (419, 423) is coated with silica (421).

38. The device (401) of any one of claims 33 to 36, wherein one of the first chamber (413) and the second chamber (415) includes an interior surface (419, 423), wherein the interior surface (419, 423) is coated with an anticoagulant.

39. The device (401) of any one of claims 33 to 38, wherein the separation element (427) comprises a plasma separation system (435).

40. The device (401) of claim 39, wherein the plasma separation system (435) comprises a microfluidic separation system.

41. The device (401) of claim 40, wherein the microfluidic separation system comprises a lateral cavity acoustic transducer system (113).

42. The device (401) of claim 41, wherein the lateral cavity acoustic transducer system (113) comprises: (a) a main channel (127); (b) a lateral cavity (121) extending from the main channel (127) along a non-orthogonal axis; and (c) a transducer (123) configured to oscillate fluid within the main channel (127) and the lateral cavity (121).

43. The device (401) of any one of claims 33 to 42, wherein the separation element (427) comprises one or more of a micro-channel (437), a filter (439), an anticoagulant, and a resin.

44. The device (401) of any one of claims 33 to 43, wherein the needle element (411) comprises a blood draw needle.

45. The device (401) of any one of claim 33 or 43, wherein the needle element (411) comprises a finger stick attachment.

46. The device (401) of any one of claims 33 to 45, comprising an identification element (441) associated with the main body (403).

47. The device (401) of claim 46, wherein the separation element (427) is configured to separate the blood sample into the first portion (429) based at least in part on the identification element (441).

48. The device (401) of any one of the claims 33 to 47, wherein the first chamber (413) is removably secured to the main body (403).

49. A method (501) comprising: (a) drawing (507) a blood sample into a device (401); (b) separating (507) the blood sample into a sample of whole blood, a sample of plasma, and a sample of serum; (c) transmitting the sample of whole blood into a first chamber (413) connected to the device (401); (d) transmitting the sample of plasma into a second chamber (415) connected to the device (401); and (e) transmitting the sample of serum into a third chamber (417) connected to the device (401).

50. The method (501) of claim 49, further comprising disconnecting one or more of the first chamber (413), the second chamber (415), and the third chamber (417) from the device (401).

51. The method (501) of claim 49 or 50, further comprising: (a) aspirating the blood sample through a needle element (411) into a separation element (427) of the device (401); and (b) transmitting the sample of blood through the separation element (427) to separate the blood sample into the sample of whole blood, the sample of plasma, and the sample of serum.

52. The method (501) of claim 51, wherein the needle element (411) comprises a blood draw needle.

53. The method (501) of claim 51, wherein the needle element (411) comprises a finger stick attachment.

54. The method (501) of any one of claims 49 to 53, further comprising coating an interior surface (419) of the first chamber (413) with an anticoagulant (425).

55. The method (501) of any one of claims 49 to 54, further comprising coating an interior surface of the third chamber (417) with silica (21).

56. The method (501) of any one of claims 49 to 55, further comprising: (a) prior to drawing the blood sample into the device (401), connecting (503) a syringe to the device (401); and (b) in response to connecting the syringe to the device (401), creating (505) a vacuum in the device (401).

57. The method (501) of claim 56, further comprising after transmitting the sample of whole blood into the first chamber (413) connected to the device (401), the sample of plasma into the second chamber (415) connected to the device (401), and the sample of serum into the third chamber (417) connected to the device (401), disconnecting the syringe from the device (401).

58. The method (501) of claim 57, further comprising after disconnecting the syringe from the device (401), loading (509) the device (401) into a sample rack.

59. The method (501) of claim 58, further comprising inserting a first portion (429) of the device (401) into a slot defined by the sample rack to load the device (401) into the sample rack, wherein the syringe is connected to the first portion (429) of the device (401).

60. A system comprising: (a) a device (401) comprising: (i) a main body (403), (ii)
a needle attachment feature (409), wherein the needle attachment feature (409) is configured to selectively couple a needle element (411) to the main body (403), wherein the needle attachment feature (409) is configured to transmit a blood sample from the needle element (411) into the main body (403), and (iii) a separation element (427) disposed inside the main body (403), wherein the separation element (427) is configured to receive the blood sample and separate the blood sample into a sample of whole blood, a sample of plasma, and a sample of serum; (b) a first sample tube (413), wherein the first sample tube is removably connected to the device (401), wherein the first sample tube is configured to receive the sample of whole blood from the device (401); (c) a second sample tube (415), wherein the second sample tube is removably connected to the device (401), wherein the second sample tube is configured to receive the sample of plasma from the device (401); and (d) a third sample tube (417), wherein the third sample tube is removably connected to the device (401), wherein the third sample tube is configured to receive the sample of serum from the device (401).

61. The system of claim 60, wherein an interior surface (419) of the first sample tube (413) includes a layer of anticoagulant (425).

62. The system of claim 60 or 61, wherein an interior surface of the third sample tube (417) includes a layer of silica (421).

63. The system of any one of claims 60 to 62, wherein the separation element (427) comprises one or more of a micro-channel (437), a filter (439), an anticoagulant, and a resin.

64. The system of any one of claims 60 to 63, wherein the separation element (427) comprises a plasma separation system (435).

65. The system of claim 64, wherein the plasma separation system (435) comprises a microfluidic separation system.

66. The system of claim 65, wherein the microfluidic separation system comprises a lateral cavity acoustic transducer system (113).

67. The system of claim 66, wherein the lateral cavity acoustic transducer system (113) comprises: (a) a main channel (127); (b) a lateral cavity (121) extending from the main channel (127) along a non-orthogonal axis; and (c) a transducer (123) configured to oscillate fluid within the main channel (127) and the lateral cavity (121).

68. The system of any one of claims 60 to 67, wherein the needle element (411) comprises a blood draw needle.

69. The system of any one of claims 60 to 67, wherein the needle element (411) comprises a finger stick attachment.

70. An analyzer (601) configured to receive a tube (603) containing a blood sample (605), the analyzer (601) comprising: (a) a separation system (607); (b) a transfer element, wherein the transfer element is configured to obtain at least a portion of the blood sample (605) from the tube (603), wherein the transfer element is configured to deposit the portion of the blood sample (605) into the separation system (607); and (c) a movement system (609), wherein the movement system (609) is configured to move the transfer element between the tube (603) and the separation system (607); and wherein the separation system (607) is configured to separate the portion of the blood sample (605) into a separated sample, wherein the separated sample comprises one of a plasma sample and a serum sample.

71. The analyzer (601) of claim 70, wherein the transfer element is a first transfer element (619) and further comprising: (a) an analysis system (611); and (b) a second transfer element (621), wherein the second transfer element (621) is configured to obtain at least a portion of the separated sample from the separation system (607), wherein the second transfer element (621) is configured to deposit the portion of the separated sample into the analysis system (611); and wherein the movement system (609) is configured to move the second transfer element (621) between the separation system (607) and the analysis system (611).

72. The analyzer (601) of claim 71, wherein the analysis system (611) comprises one of a spectroscopic analyzer, a thermocycler element for PCR, an isotheral amplification element, an immune assay, and an Elisa system.

73. The analyzer (601) of claim 71 or 72, wherein one of the first transfer element (619) and the second transfer element (621) comprises a pipette.

74. The analyzer (601) of claim 70 or 71, wherein the separation system (607) comprises a microfluidic separation system.

75. The analyzer (601) of claim 74, wherein the microfluidic separation system comprises a lateral cavity acoustic transducer system (113).

76. The analyzer (601) of claim 75, wherein the lateral cavity acoustic transducer system (113) comprises: (a) a main channel (127); (b) a lateral cavity (121) extending from the main channel (127) along an axis; and (c) a transducer (123) configured to oscillate fluid within the main channel (127) and the lateral cavity (121).

77. The analyzer (601) of any one of claims 70 to 76, wherein the separation system (607) defines a cassette receptacle (615), wherein the cassette receptacle (615) is configured to receive a cassette (617) therein.

78. An analyzer system comprising: (a) a cassette (617); and (b) an analyzer (601) configured to receive a tube (603) containing a blood sample (605) and the cassette (617), the analyzer (601) comprising: (i) a separation system (607); (ii) a transfer element, wherein the transfer element is configured to obtain at least portion of the blood sample from the tube (603), wherein the transfer element is configured to deposit the portion of the blood sample into the separation system (607); (iii) a movement system (609), wherein the movement system (609) is configured to move the transfer element between the tube (603) and the separation system (607); and (iv) a cassette receptacle (615) defined by the separation system (607), wherein the cassette receptacle (615) is configured to removably receive the cassette (617) therein; and wherein the separation system (607) is configured to separate the portion of the blood sample into a separated sample via the cassette (617), wherein the separated sample comprises one of a plasma sample and a serum sample.

79. The analyzer system of claim 78, wherein the cassette (617) comprises a plurality of separation channels, wherein the separation system (607) is configured to separate the portion of the blood sample into the separated sample via one of the plurality of separation channels.

80. The analyzer system of claim 79, wherein each separation channel (623) in the plurality of separation channels comprises: (a) a first portion (625), wherein the first portion (625) is configured to receive the portion of the blood sample therein; and (b) a second portion (627), wherein the second portion (627) is configured to receive the portion of the blood sample from the first portion (625) and separate the portion of the blood sample into the separated sample.

81. The analyzer system of claim 80, wherein the second portion (627) comprises a microfluidic separation system.

82. The analyzer system of claim 81, wherein the microfluidic separation system comprises a lateral cavity acoustic transducer system (113).

83. The analyzer system of claim 82, wherein the lateral cavity acoustic transducer system (113) comprises: (a) a main channel (127); (b) a lateral cavity (121) extending from the main channel (127) along an axis; and (c) a transducer (123) configured to oscillate fluid within the main channel (127) and the lateral cavity (121).

84. A method (701) of analyzing a blood sample in a tube (603), the method (601) comprising: (a) placing (703) the tube (603) into an analyzer (601); (b) transferring (705) at least a portion of the blood sample from the tube (603) into a separation system (607) of the analyzer (601), wherein the transferring (705) is performed automatically within the analyzer; (c)
separating (707) the portion of the blood sample into a separated sample by the separation system (607), wherein the separated sample is one of a plasma sample and a serum sample; (d)
transferring (709) the separated sample into an analysis system (611), wherein the transferring is performed automatically within the analyzer; and (e) analyzing (711) the separated sample via the analysis system (611).

85. The method (701) of claim 84, wherein the separation system (607) comprises a removable cassette (617), and further comprising transferring the portion of the blood sample from the tube (603) into the cassette (617).

86. The method (701) of claim 85, wherein the cassette (617) comprises at least one sealed well, and further comprising: (a) puncturing one of the at least one sealed wells to form a punctured well; and (b) transferring the portion of the blood sample from the tube (603) into the punctured well.

87. The method (701) of claim 85 or 86, wherein the cassette (617) comprises a separation portion and at least one open well, and further comprising moving the portion of the blood sample from the punctured well into the separation portion and toward one of the at least one open wells to separate the portion of the blood sample into the separated sample.

88. The method (701) of any one of claims 84 to 87, wherein the separation system (607) comprises a microfluidic separation system.

89. The method (701) of claim 88, wherein the microfluidic separation system comprises a lateral cavity acoustic transducer system (113).

90. The method (701) of claim 89, wherein the lateral cavity acoustic transducer system (113) comprises: (a) a main channel (127); (b) a lateral cavity (121) extending from the main channel (127) along an axis; and (c) a transducer (123) configured to oscillate fluid within the main channel (127) and the lateral cavity (121).

91. The method (701) of claim 85, further comprising: (a) transferring (705) the portion of the blood sample from the tube (603) into a first portion (625) of the cassette (617); (b) separating (707) the portion of the blood sample into the separated sample in a second portion (627) of the cassette (617); and (c) transferring (709) the separated sample from a third portion (629) of the cassette (617) into the analysis system (611).

92. The method (701) of claim 91, further comprising puncturing the first portion (625) prior to transferring (705) the portion of the blood sample from the tube (603) into the first portion (625) of the cassette (617).

93. The method (701) of claim 91 or 92, wherein the second portion (627) comprises a microfluidic separation system.

94. The method (701) of claim 85, wherein the cassette (617) comprises a plurality of separation channels, wherein each separation channel (623) in the plurality of separation channels comprises a first portion (625) and a second portion (627), and further comprising: (a) selecting a separation channel (623) from the plurality of separation channels; (b)
transferring the portion of the blood sample from the tube (603) into the first portion (625) of the selected separation channel (623); and (c) separating the portion of the blood sample into the separated sample in the second portion (627) of the selected separation channel (623).

95. The method (701) of claim 94, wherein each separation channel (623) in the plurality of separation channels comprises a third portion (629), and further comprising transferring the separated sample from the third portion (629) of the selected separation channel into the analysis system (611).

96. The method (701) of any one of claims 91 to 94, further comprising puncturing the first portion (625) of the selected separation channel (623) prior to transferring the portion of the blood sample from the tube (603) into the first portion (625) of the selected separation channel (623).

97. The method (701) of any one of claims 91 to 96, wherein the second portion (627) comprises a microfluidic separation system.

98. The method (701) of any one of claims 85 to 97, further comprising manually removing the cassette (617) from the analyzer.

99. The method of claim 84, further comprising separating the portion of the blood sample into multiple separated samples by the separation system (607).

100. The method of claim 99, further comprising transferring a first separated sample in the multiple separated samples into a chemistry analyzer, wherein the transferring of the first separated sample is performed automatically within the chemistry analyzer.

101. The method of claim 99 or 100, further comprising transferring a second separated sample in the multiple separated samples into an immunoassay analyzer, wherein the transferring of the second separated sample is performed automatically within the immunoassay analyzer.

102. The method of any one of claims 99 to 101, further comprising transferring a third separated sample in the multiple separated samples into a molecular analyzer, wherein the transferring of the third separated sample is performed automatically within the molecular analyzer.

103. The method of any one of claims 99 to 102, further comprising transferring a fourth separated sample in the multiple separated samples into a mass spectrometry analyzer, wherein the transferring of the fourth separated sample is performed automatically within the mass spectrometry analyzer.

104. The method of claim 94, further comprising transferring the second portion of the blood sample from the tube (603) into a storage container.

105. The method of any one of claims 84 to 98, further comprising transferring at least portion of the separated sample into a storage container.

106. A cassette (617) configured to be selectively disposed in an analyzer (601), the cassette (617) comprising: (a) a main body (613); and (b) at least one separation channel (623) disposed within the main body (613), wherein each separation channel (623) is configured to separate at least a portion of a blood sample into a separated sample.

107. The cassette (617) of claim 106, wherein each separation channel (623) comprises: (a) a first portion (625), wherein the first portion (625) is configured to receive the portion of the blood sample therein; and (b) a second portion (627), wherein the second portion (627) is configured to receive the portion of the blood sample from the first portion (625) and separate the portion of the blood sample into the separated sample.

108. The cassette (617) of claim 107, wherein the second portion (627) comprises a microfluidic separation system.

109. The cassette (617) of claim 108, wherein the microfluidic separation system comprises a lateral cavity acoustic transducer system (113).

110. The cassette (617) of claim 109, wherein the lateral cavity acoustic transducer system (113) comprises: (a) a main channel (127); (b) a lateral cavity (121) extending from the main channel (127) along an axis; and (c) a transducer (123) configured to oscillate fluid within the main channel (127) and the lateral cavity (121).

111. The cassette (617) of any one of claims 107 to 110, wherein the first portion (625) is a sealed well configured to be punctured prior to receiving the portion of the blood sample therein.

112. The cassette (617) of any one of claims 107 to 111, wherein each separation channel (623) comprises a third portion (629) configured to receive the separated sample from the second portion (627).

113. The cassette (617) of claim 112, wherein the third portion (629) is an open well.

114. The cassette (617) of any one of claims 106 to 113, wherein the separated sample is at least 10 microliters.

115. An analyzer system comprising: (a) a transducer (825); (b) an LCAT chip (800, 841) configured to receive a whole blood sample (605) and produce a plasma sample in response to stimulation by the transducer (632); and (c) an LCAT rack (801, 845), wherein the LCAT rack (801, 845) is configured to removably receive the LCAT chip (800, 841) therein; and wherein the transducer (632) is configured to stimulate the LCAT chip (800, 841) to produce plasma while the LCAT chip (800, 841) is in the LCAT rack (801, 845).

116. The analyzer system of claim 115, the LCAT chip (800, 841) comprising an input well (809, 851) configured to receive the whole blood sample (605) therein.

117. The analyzer system of claim 116, the LCAT rack (801, 845) defining an input well receiving space (817, 858), wherein the input well (809, 851) is disposed within the input well receiving space (817, 858) when the LCAT chip (800, 841) is disposed within the LCAT rack (801, 845).

118. The analyzer system of any one of claims 115 to 117, the LCAT chip (800, 841) comprising an output well (811) configured to collect the plasma generated therein.

119. The analyzer system of claim 118, the LCAT rack (801, 845) defining an output well receiving space (819), wherein the output well (811) is disposed within the output well receiving space (819) when the LCAT chip (800, 841) is disposed within the LCAT rack (801, 845).

120. The analyzer system of any one of claims 115 to 119, the LCAT chip (800, 841) comprising a dispense tip (857) configured to expel the plasma generated therein.

121. The analyzer system of any one of claims 115 to 120, the LCAT chip (800, 841) comprising a housing (802, 855) defining a notch (815, 859), the LCAT rack (801, 845) comprising an alignment feature, wherein the notch (815, 859) and the alignment feature align when the LCAT chip (800, 841) is fully inserted into the LCAT rack (801, 845).

122. The analyzer system of any one of claims 115 to 121, the LCAT rack (801, 845) defining a chip slot (856) therein, wherein the LCAT chip (800, 841) is disposed in the chip slot (856) when the LCAT chip (800, 841) is disposed within the LCAT rack (801, 845).

123. The analyzer system of any one of claims 115 to 122, wherein the transducer (825) is disposed in a plasma separation module (821), wherein the plasma separation module (821) defines a transducer slot (823, 871) sized to receive the LCAT rack (801, 845).

124. The analyzer system of any one of claims 115 to 122, wherein the transducer (825) is disposed in the LCAT rack (801, 845).

125. The analyzer system of any one of claims 115 to 122, wherein the transducer (825) is disposed within a transducer cartridge (843), wherein the LCAT rack (801, 845) is configured to removably receive the transducer cartridge (843) therein.

126. The analyzer system of claim 125, wherein the transducer cartridge (843) comprises a lead (875), wherein the lead (875) is operably coupled with the transducer (825).

127. The analyzer system of claim 126, wherein the LCAT rack (801, 845) defines a lead slot (879), wherein the lead (875) is disposed within the lead slot (879) when the transducer cartridge (843) is disposed within the LCAT rack (801, 845).

128. The analyzer system of claim 126 or 127, further comprising a transducing module (881), wherein the transducing module (881) is configured to provide electrical power to the lead (875) of the transducer cartridge (843).

129. The analyzer system of any one of claims 115 to 128, the transducer (825) further comprising piezo electric material (873).

IX. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various versions and embodiments in the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

What is claimed is:

1. A collection system (1) comprising:
(a) a tube (102); and
(b) a holder (104), wherein the holder (104) is configured to receive the tube (102) therein.

2. The collection system (1) of claim 1, further comprising a plasma separation system (114).

3. The collection system (1) of claim 2, wherein the plasma separation system (114) comprises a microfluidic separation system.

4. The collection system (1) of claim 3, wherein the microfluidic separation system comprises a lateral cavity acoustic transducer system (113).

5. The collection system (1) of claim 4, wherein the lateral cavity acoustic transducer system (113) comprises:
(a) a main channel (127);
(b) a lateral cavity (121) extending from the main channel (127) along a non-orthogonal axis; and
(c) a transducer (123) configured to oscillate fluid within the main channel (127) and the lateral cavity (121).

6. The collection system (1) of any one of claims 1 to 5, wherein the tube (102) defines a collection chamber (112).

7. The collection system (1) of claim 6, wherein the collection chamber (112) defines an analysis pocket (116) therein.

8. The collection system (1) of any one of claims 1 to 7, wherein the holder (104) comprises an imaging system.

9. The collection system (1) of claim 8, wherein an optics element of the imaging system is directed at the analysis pocket (116) when the tube (102) is disposed in the holder (104).

10. The collection system (1) of any one of claims 7 to 9, wherein a portion of the tube (102) adjacent the analysis pocket (116) is transparent.

11. The collection system (1) of any one of claims 6 to 10, wherein the holder (104) comprises a reagent reservoir (138), wherein the holder (104) is configured to transfer a reagent from the reagent reservoir (138) to the collection chamber (112).

12. The collection system (1) of any one of claims 1 to 10, wherein the tube (102) comprises a label area (120) on an outer surface.

13. The collection system (1) of claim 12, wherein the holder (104) comprises a labeling system (142), wherein the labeling system (142) is configured to apply a label (122) to the label area (120) when the tube (102) is disposed in the holder (104).

14. The collection system (1) of claim 12 or 13, wherein the label area (120) comprises a flat rectangular-shaped surface.

15. The collection system (1) of any one of the claims 1 to 13, wherein the tube (102) comprises a tube alignment element (124), wherein the holder (104) includes a holder alignment element (130), wherein the tube alignment element (124) is configured to cooperate with the holder alignment element (130) when the tube (102) is disposed in the holder (104).

16. A collection system (1) comprising:
(a) a tube (102) comprising:
(i) a collection chamber (112),
(ii) a plasma separation system (114), wherein the plasma separation system (114) is disposed in the collection chamber (112), and
(iii) an analysis pocket (116) extending from the collection chamber (112), wherein the analysis pocket (116) is configured to receive therein a unit of plasma provided from the plasma separation system (114); and
(b) a holder (104) configured to draw power from a power supply (148), the holder (104) comprising:
(i) a display (134), wherein the display (134) is powered by the power supply (148),
(ii) a tube receptacle (126), wherein the tube receptacle (126) is configured to receive the tube (102) therein,
(iii) an analysis system (132), wherein the analysis system (132) is configured to collect a set of data from the unit of plasma disposed in the analysis pocket (116), wherein the analysis system (132) is configured to actuate the display (134) to display the set of data,
(iv) a labeling system (142), wherein the labeling system (142) is configured to label the tube (102), and
(v) a transducer (123) configured to oscillate fluid within a main channel (127) and a lateral cavity (121) of the plasma separation system (114).

17. The collection system (1) of claim 16, wherein the plasma separation system (114) comprises a microfluidic separation system.

18. The collection system (1) of claim 17, wherein the microfluidic separation system comprises a lateral cavity acoustic transducer system (113).

19. The collection system (1) of any one of claims 16 to 18, the holder (104) comprising a reagent reservoir (138), wherein the holder (104) is configured to transfer a reagent from the reagent reservoir (138) to the collection chamber (112).

20. A method (300) comprising:
(a) placing (302) a blood sample into a tube (102);
(b) receiving (306) the tube (102) in a holder (104);
(c) oscillating the blood sample in the tube (102) with a transducer (123);
(d) in response to oscillating the blood sample in the tube (102) with the transducer (123), creating (304) a plasma sample from the blood sample within the tube (102); and
(e) conducting (308) spectroscopic or imaging analysis on the plasma sample with an analysis system (132) of the holder (104) to derive a sample quality.

21. The method (300) of claim 20, further comprising indicating (310) the sample quality to a user on a display screen (134) of the holder (104).

22. The method (300) of claim 20 further comprising indicating the sample quality to a user on an indication system of the holder (104).

23. The method (300) of any one of claims 20 to 22, further comprising providing the sample quality to a central analysis platform.

24. The method (300) of any one of claims 20 to 23, further comprising labeling (316) the tube (102) while the tube (102) is disposed within the holder (104).

25. The method (300) of any one of claims 20 to 24, further comprising programming an RFID chip embedded within the tube (102) while the tube (102) is disposed within the holder (104).

26. The method (300) of any one of claims 20 to 25, further comprising creating the plasma sample via a plasma separation system (114) disposed in the tube (102).

27. The method (300) of claim 26, wherein the plasma separation system (114) comprises a microfluidic separation system.

28. The method (300) of claim 27, wherein the microfluidic separation system comprises a lateral cavity acoustic transducer system (113).

29. The method (300) of any one of claims 20 to 28, wherein the transducer (123) is disposed in the holder (104).

30. The method (300) of any one of claims 20 to 29, wherein the tube (102) comprises an electronic label.

31. The method (300) of claim 30, wherein the holder (104) comprises a labeling system (142), wherein the labeling system (142) is configured to program the electronic label when the tube (102) is disposed in the holder (104).

32. The method (300) of claim 20, further comprising indicating (310) the sample quality to a user.
